(19) United States
(12) Patent Application Publication
(10) Pub. No.: US 2024/0182513 A1
(43) Pub. Date: Jun. 6, 2024

(54) SEPARATION OF METAL IONS FROM A SAMPLE USING GLYCOLIPIDS

(71) Applicant: ARIZONA BOARD OF REGENTS ON BEHALF OF THE UNIVERSITY OF ARIZONA, Tucson, AZ (US)

(72) Inventors: David Hogan, Tucson, AZ (US); Raina M. Maier, Tucson, AZ (US); Jeanne E. Pemberton, Tucson, AZ (US)

(21) Appl. No.: 18/394,478

(22) Filed: Dec. 22, 2023

Related U.S. Application Data
(63) Continuation-in-part of application No. PCT/US22/35869, filed on Jun. 30, 2022.
(60) Provisional application No. 63/217,149, filed on Jun. 30, 2021.

(51) Int. Cl. C07H 23/00; B09C 1/08; C02F 1/00; C02F 1/24; C02F 1/38; C02F 1/54; C02F 101/20; C02F 103/06; G01N 1/40; G21F 9/06

(52) U.S. Cl. CPC C07H 23/00; B09C 1/08; C02F 1/001; C02F 1/24; C02F 1/38; C02F 1/547; G01N 1/4077; G21F 9/06; C02F 2101/20; C02F 2103/06; G01N 2001/4088

(57) ABSTRACT

Glycolipid-heavy metal ion complexes, compositions useful for forming glycolipid-heavy metal ion complexes, and methods for separating metal ion(s) from a sample using glycolipids are described herein.

### CROSS-REFERENCE TO RELATED APPLICATIONS

This application is a continuation-in-part and claims benefit of PCT Application No. PCT/US22/35869 filed Jun. 30, 2022, which claims benefit of U.S. Provisional Application No. 63/217,149, filed Jun. 30, 2021, the specification(s) of which is/are incorporated herein in their entirety by reference.

### STATEMENT REGARDING FEDERALLY SPONSORED RESEARCH OR DEVELOPMENT

This invention was made with government support under Grant Nos. P42 ES004940 and R43 ES029423 awarded by the National Institutes of Health and Grant Nos. 0714245 and 1339597 awarded by the National Science Foundation. The government has certain rights in the invention.

### FIELD OF THE INVENTION

The present invention provides a method for separating metal ion(s) from a sample using glycolipids.

### BACKGROUND OF THE INVENTION

Mining of uranium for defense-related purposes as well as industrial waste has left a substantial legacy of water and soil pollution that threatens human and environmental health. Contaminated waters in the arid southwest are of particular concern, as water resource demand and water scarcity issues become more pronounced. In addition, soil contamination due to mining and industrial processes resulting in heavy metal waste has rendered many parts of the land environmentally hazardous. The development of remediation strategies to treat impacted waters that contain metal ions, such as uranium as well as other heavy metals, including lanthanides and actinides, will become increasingly vital to meet future water needs.

For example, from 1947 to 1970, 75.9 million tons of uranium ore were mined from 4,225 mines across 19 states in the United States to fulfill defense-related purposes. Development of these uranium resources has left a substantial legacy of pollution that threatens human and environmental health due to radiological and physical hazards, ecological degradation, and water quality degradation; of the 4,225 sites identified, only 15% have undergone some form of reclamation or remediation. Degradation of water quality in the desert southwest is of special concern due to persistent drought conditions and growing populations dependent on limited water resources. As an example, in the Four Corners Region, the Navajo Nation alone contains 523 abandoned uranium mining sites. Of those sites, 518 are within one mile of a perennial or intermittent surface water source, and 58 are located within a quarter mile of human and livestock drinking water wells. Furthermore, 12.8% of tested water sources on the Nation exceed national drinking water standards for uranium.

In addition to water contamination, soils on many land areas, such as military installations and industrial sites, are often contaminated with heavy metal ions (e.g., Cr, Cu, Zn, Pb, Cd, Ni). In fact, a survey of military installations shows that soils contaminated by heavy metal ions are a common problem. Over 50 percent of the installations surveyed have potential heavy metal problems that may prove to be costly and/or beyond current technology to remediate. Heavy metal ions most often found as contaminants in military installments include chromium, lead, arsenic, and cadmium.

Furthermore, the smelting process in metal mines also produces large amounts of waste, resulting in a high accumulation of heavy metals in the soil and in the river and underground water surrounding the mining area. Similarly, the levels of heavy metal ion contamination in the soil, air, river, and crops from mining-affected areas are reportedly higher than those in non-mining areas in many countries. In addition to causing environmental damage, heavy metal ions enter plants by absorption, e.g., through the vegetable roots. These heavy metal ions often enter into the food chain, and high consumption of contaminated vegetables can pose a serious risk to animals, including humans.

While methods exist for the remediation of heavy metal ions from water and/or soil, these methods are often costly and can generate other undesired by-products.

Therefore, there is a need for a green-chemistry based remediation of water, soil, and other environmental sources that are contaminated with heavy metals.

### BRIEF SUMMARY OF THE INVENTION

It is an objective of the present invention to provide compositions and methods that allow for separating metal ion(s) from a sample (e.g., an environmental sample) using glycolipids, as specified in the independent claims. Embodiments of the invention are given in the dependent claims. Embodiments of the present invention can be freely combined with each other if they are not mutually exclusive.

Some aspects of the invention are based on the discovery by the present inventors that glycolipids can be used as green biosurfactants to bind or coordinate heavy metals, thereby allowing easy removal of heavy metals from samples. Unless the context requires otherwise, the terms “heavy metal” and “heavy metal ion” are used interchangeably herein to refer to heavy metal ions that can form a coordinated complex with glycolipids of the invention.

In one particular aspect of the invention, a method is provided for removing a heavy metal from a sample. The sample can be water, soil, industrial waste, or a combination thereof. The method generally comprises: i) contacting said sample with an aqueous solution comprising a glycolipid to form a glycolipid-heavy metal ion complex; and ii) separating said glycolipid-heavy metal ion complex, thereby removing said heavy metal ion from said sample.

In some embodiments, said sample comprises soil, groundwater, industrial wastewater, acid mine drainage, mining (both coal and hard rock) process waters and solid residuals, produced waters, electroplating solutions, coal combustion process waters, landfill leachates, e-waste, and fly ash.

Yet in other embodiments, said heavy metal comprises uranium, Y, Ce, Pr, Nd, Pm, Sm, Eu, Gd, Tb, Dy, Ho, Er, Tm, Yb, Lu, La, Cu, Ag, Au, Pd, Pt, Pb, Cd, Zn, Tl, Hg, or a combination thereof.

Still, in other embodiments, the glycolipid is of the formula:

A-B

wherein A is selected from the group consisting of a monosaccharide, a disaccharide, and a derivative thereof; and B is a moiety of the formula:

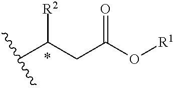

wherein * is a chiral center; R² is C₁-C₂₀ hydrocarbon optionally having 1, 2, or 3 carbon-carbon double bonds; and R¹ is H, C₁-C₂₀ hydrocarbon optionally having 1, 2, or 3 carbon-carbon double bonds, or a moiety of the formula:

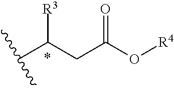

where * is a chiral center, R³ is C₁-C₂₀ hydrocarbon optionally having 1, 2, or 3 carbon-carbon double bonds, and R⁴ is H or C₁-C₂₀ hydrocarbon optionally having 1, 2, or 3 carbon-carbon double bonds.

In another aspect of the invention, a glycolipid-heavy metal ion complex is provided, said glycolipid-heavy metal ion complex comprising: at least one glycolipid of the formula:

A-B

wherein A is selected from the group consisting of a monosaccharide, a disaccharide, and a derivative thereof; and B is a moiety of the formula:

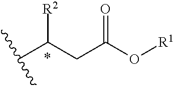

wherein * is a chiral center; R² is C₁-C₂₀ hydrocarbon optionally having 1, 2, or 3 carbon-carbon double bonds; and R¹ is H, C₁-C₂₀ hydrocarbon optionally having 1, 2, or 3 carbon-carbon double bonds, or a moiety of the formula:

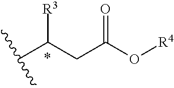

wherein R³ is C₁-C₂₀ hydrocarbon optionally having 1, 2, or 3 carbon-carbon double bonds, and R⁴ is H or C₁-C₂₀ hydrocarbon optionally having 1, 2, or 3 carbon-carbon double bonds; and a heavy metal ion, wherein said heavy metal ion comprises an ion of uranium, Y, Ce, Pr, Nd, Pm, Sm, Eu, Gd, Tb, Dy, Ho, Er, Tm, Yb, Lu, La, Cu, Ag, Au, Pd, Pt, Pb, Cd, Zn, Tl, Hg, or a combination thereof; wherein the at least one glycolipid and the heavy metal ion together form the glycolipid-heavy metal ion complex.

In yet another aspect of the invention, a composition is provided, said composition comprising at least one glycolipid of the formula:

A-B

wherein A is selected from the group consisting of a monosaccharide, a disaccharide, and a derivative thereof, and B is a moiety of the formula:

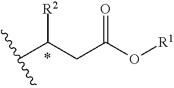

wherein * is a chiral center; R² is C₁-C₂₀ hydrocarbon optionally having 1, 2, or 3 carbon-carbon double bonds; and R¹ is H, C₁-C₂₀ hydrocarbon optionally having 1, 2, or 3 carbon-carbon double bonds, or a moiety of the formula:

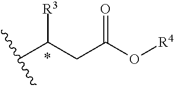

wherein R³ is C₁-C₂₀ hydrocarbon optionally having 1, 2, or 3 carbon-carbon double bonds, and R⁴ is H or C₁-C₂₀ hydrocarbon optionally having 1, 2, or 3 carbon-carbon double bonds; a frothing agent, wherein the frothing agent comprises a C1-C6 alcohol; and water.

It should be appreciated that when R¹, R², R³, or R⁴ contains 1, 2, or 3 carbon-carbon double bonds, the number of carbon atoms is at least 2, 4, and 6, respectively. That is, R¹, R², R³, or R⁴ can be C₁₋₂₀ alkyl or C₂₋₂₀ alkenyl (when one carbon-carbon double bond is present), C₄₋₂₀ alkenyl (when two carbon-carbon double bonds are present), or C₆₋₂₀ alkenyl (when three carbon-carbon double bonds are present). The term “alkyl” refers to a monovalent saturated linear monovalent hydrocarbon moiety of one to thirty, typically one to twenty, often two to twenty, and more often six to twenty carbon atoms, or a saturated branched monovalent hydrocarbon moiety of three to thirty, typically six to twenty, and often six to eighteen carbon atoms. Exemplary nonpolar alkyl groups include but are not limited to, hexyl, decyl, dodecyl, tetradecyl, hexadecyl, octadecyl, and the like. The term “alkenyl” means a linear monovalent hydrocarbon moiety or a branched monovalent hydrocarbon moiety containing at least one carbon-carbon double bond.

In some embodiments, B is a moiety of the formula:

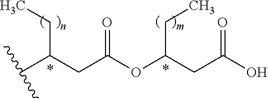

where each of m and n is independently an integer from 0 to 19, or 0 to 14, and each of * is independently a chiral center.

In one particular embodiment, A is a monosaccharide or a thiol derivative thereof. Still, in another embodiment, B is attached to the hydroxyl group of the anomeric carbon or a thiol derivative thereof of said monosaccharide.

Still, in further embodiments, said monosaccharide is selected from the group consisting of glucose, fructose, galactose, rhamnose, arabinose, xylose, fucose, mannose, ribose, lyxose, allose, altrose, gulose, idose, talose, and a thiol derivative thereof.

Yet, in other embodiments, A is a disaccharide or a thiol derivative thereof. In some embodiments, said disaccharide comprises a 1,4-linkage or a 1,6-linkage between two monosaccharides. Still, in other embodiments, said disaccharide is selected from the group consisting of lactose, maltose, melibiose, cellobiose, rutinose, sucrose, trehalose, and a thiol derivative thereof.

In further embodiments, R1 is a moiety of the formula:

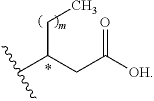

In some instances within these embodiments, m is independently an integer from 2 to 14. In some particular embodiments, m is 6, 8, or 10.

In certain embodiments, R1 is C₆₋₂₀ alkyl.

In some embodiments of the method, the step of separating said glycolipid-heavy metal ion complex comprises ion flotation and/or precipitate flotation of said glycolipid-heavy metal ion complex.

Yet in other embodiments of the method, said step of separating said glycolipid-heavy metal ion complex comprises precipitation of said glycolipid-heavy metal ion complex through gravitational settling.

In certain embodiments of the method, said step of separating said glycolipid-heavy metal ion complex comprises centrifugation.

In certain embodiments of the method, said step of separating said glycolipid-heavy metal ion complex comprises filtration.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a structural formula of certain embodiments of monorhamnolipids, wherein values of m and n are each independently in a range from 0 to 19.

FIG. 2 shows a schematic diagram of a flotation apparatus.

FIG. 3 shows the results of modeling uranium speciation over a range of pH values.

FIG. 4 shows the removal of uranium from solution samples using a biosynthetic monorhamnolipid over a range of pH values.

FIG. 5A and 5B show the removal of uranium from solution samples using synthetic monorhamnolipids: FIG. 5A shows results when flotation was performed on a pH 7 solution; FIG. 5B shows results when flotation was performed on a pH 6.5 solution.

FIG. 6 shows the proposed complexation of monorhamnolipids to uranyl species.

FIG. 7 shows the flotation results using a dirhamnolipid.

FIG. 8 shows the results over time for ion flotation of a variety of rare earth elements using a synthetic monorhamnolipid. Bars from left to right represent 0 min, 5 min, 10 min, 15 min, 20 min, and 25 min, respectively.

FIG. 9 shows the precipitation of La with synthetic monorhamnolipid at a ratio of 5:1 after mixing (left) and centrifugation (right) with the respective removal efficiencies shown in the bottom left of the image.

FIG. 10 shows metal concentrations (mmol/L) of lead in the reaction tube initially and after the three treatments. Cᵢ stands for the initial metal concentration, Cₙₜ stands for the metal concentration after the rhamnolipid and no additional treatment, C_{f} stands for the metal concentration after the rhamnolipid and filtration treatment, Cₒ stands for the metal concentration after rhamnolipid and centrifugation treatment. The efficiency of removal is presented as a percent (calculated in Equations 2-4) below the concentration. Cells highlighted in dark green indicate removal >95%, light green indicates 85% to 94.5%, and yellow indicates 70% to 84.9%.

FIG. 11 shows metal concentrations (mmol/L) of lanthanum in the reaction tube initially and after the three treatments. Cᵢ stands for the initial metal concentration, Cₙₜ stands for the metal concentration after the rhamnolipid and no additional treatment, C_{f} stands for the metal concentration after the rhamnolipid and filtration treatment, Cₒ stands for the metal concentration after rhamnolipid and centrifugation treatment. The efficiency of removal is presented as a percent (calculated in Equations 2-4) below the concentration. Cells highlighted in dark green indicate removal >95%, light green indicates 85% to 94.5%, and yellow indicates 70% to 84.9%.

FIG. 12 shows metal concentrations (mmol/L) of magnesium in the reaction tube initially and after the three treatments. Cᵢ stands for the initial metal concentration, Cₙₜ stands for the metal concentration after the rhamnolipid and no additional treatment, C_{f} stands for the metal concentration after the rhamnolipid and filtration treatment, Cₒ stands for the metal concentration after rhamnolipid and centrifugation treatment. The efficiency of removal is presented as a percent (calculated in Equations 2-4) below the concentration. Cells highlighted in dark green indicate removal >95%, light green indicates 85% to 94.5%, and yellow indicates 70% to 84.9%.

FIG. 13 shows conditional stability (metal binding) constants, molar ratios, and statistical analysis for metal complexes with glycolipids. The measured glycolipid-metal binding constants (Log β), standard error, and 95% confidence intervals are shown for a variety of glycolipids that were produced synthetically. In addition, the molar ratios (χ, expressed as glycolipid to metal) are provided with standard error and 95% confidence intervals. Three metals were tested, lead (Pb²⁺), zinc (Zn²⁺), and nickel (Ni²⁺). For comparison, the metal binding constants for biologically produced monorhamnolipid are provided in bold. ¹RhaC10C10=rhamnosyl-β-hydroxydecanoyl-β-hydroxydecanoate; RhaC8C8=rhamnosyl-β-hydroxyoctanoyl-β-hydroxyoctanoate; RhaC12C12=rhamnosyl-β-hydroxydodecanoyl-62-hydroxydodecanoate; RhaC14C14=rhamnosyl-β-hydroxytetradecanoyl-β-hydroxytetradecanoate; XyloC10C10=xylosyl-β-hydroxydecanoyl-β-hydroxydecanoate, GalC10C10=galactosyl-β-hydroxydecanoyl-β-hydroxydecanoate; ^{a} Values are from: Hogan, D. E., J. E. Curry, J. E. Pemberton.

### DETAILED DESCRIPTION OF THE INVENTION

Some aspects of the invention provide a composition and a method for removing heavy metal contaminants from a sample. In particular, methods of the invention include treating a sample with a glycolipid that is capable of forming a complex or coordinating with the heavy metal that is present in the sample. The glycolipid-heavy metal complex that is formed can be removed or separated from the treated sample to provide a purified sample and an isolated glycolipid-heavy metal complex. As used herein, the term “heavy metal” includes transition metals and rare earth metals, such as lanthanides and actinides. The term “heavy metal” includes rare earth metals (e.g., Y, Ce, Pr, Nd, Pm, Sm, Eu, Gd, Tb, Dy, Ho, Er, Tm, Yb, Lu, La), valuable metals (e.g., Cu, Ag, Au, Pd, Pt), and metals of environmental concern (e.g., Pb, Cd, Zn, Tl, Hg). As can be appreciated, in addition to the remediation of samples, methods of the invention can be used to recover other metals (i.e., metals not encompassed by the term “heavy metal” as described herein) from the sample.

Ion flotation is a separation process that removes metal ions from solution using air bubbles and surfactants. In some embodiments, the invention provides simple, environmentally friendly methods for removing metal ions from a sample, e.g., removing uranium from uranium-contaminated waters. Without being bound by any theory, it is believed that the basis of the ion flotation separation process is the accumulation of an appropriately charged surfactant (i.e., collector) at the air-water interface of bubbles introduced into the system. The hydrophilic, charged headgroup of these surfactants attracts surface-inactive metal ions, whereupon both are transported from the bulk solution to the surface, and the metal-collector product is concentrated into a small volume of collectible foam or scum. The concentrate can then be disposed of properly or regenerated or recovered as a useful product. Some of the benefits of ion flotation over conventional metal remediation approaches (e.g., membrane filtration, chemical precipitation, adsorbents) include but are not limited to, high metal selectivity and removal efficiency from very dilute solutions while maintaining low detention periods, space requirements, sludge volumes, and operating and energy costs.

Depending on the glycolipids used, methods of the invention allow separation of glycolipid-heavy metal ion complex (sometimes referred to simply as glycolipid-metal complex) using an ion flotation process or by collecting the precipitated glycolipid-metal complex by gravitational settling, centrifugation or filtration.

In some embodiments, the glycolipids of the invention are of the formula:

A-B (Formula I),

wherein A is selected from the group consisting of a monosaccharide, a disaccharide, and a derivative thereof; and B is a moiety of the formula:

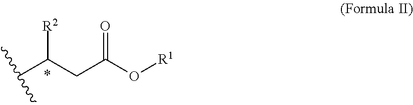

wherein * is a chiral center; R² is C₁-C₂₀ hydrocarbon optionally having 1, 2, or 3 carbon-carbon double bonds; and R¹ is H, C₁-C₂₀ hydrocarbon optionally having 1, 2, or 3 carbon-carbon double bonds, or a moiety of the formula:

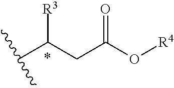

where * is a chiral center, R³ is C₁-C₂₀ hydrocarbon optionally having 1, 2, or 3 carbon-carbon double bonds, and R⁴ is H or C₁-C₂₀ hydrocarbon optionally having 1, 2, or 3 carbon-carbon double bonds.

In certain embodiments, B is a moiety of the formula:

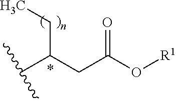

wherein R¹ is H, C₁-C₂₀ hydrocarbon optionally having 1, 2, or 3 carbon-carbon double bonds, or a moiety of the formula:

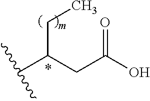

each of m and n is independently an integer from 0 to 19; and each of * is independently a chiral center.

In some embodiments, R¹ is C₆₋₂₀ alkyl, typically C₆₋₁₈ alkyl, and often C₆₋₁₄ alkyl.

Yet in other embodiments, R¹ is a moiety of the formula:

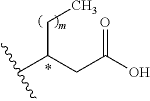

wherein m and * are as defined herein. Within these embodiments, in some instances, each of m and n is independently an integer from 0 to 19, from 4 to 16, typically from 6 to 16, often 6 to 14, and more often 6 to 12. In one particular embodiment, each of m and n is independently 6, 8, or 10.

Still, in other embodiments, the total number of carbon atoms represented by m and R1 ranges from ten to fifty, typically from twelve to forty, often from sixteen to forty, and more often from eighteen to forty.

The term “derivative” refers to any chemical modification of the parent compound or a compound derived from the parent compound. For example, a derivative of a carbohydrate includes an alkylated carbohydrate, replacement of one or more hydroxyl groups with hydrogen, halide, amine, or a thiol; modification of a hydroxyl group (e.g., by esterification, etherification, protection, etc.); as well as other derivatives known to one skilled in the art. The term carbohydrate includes pyranose and furanose carbohydrates. Exemplary derivatives of carbohydrates include, but are not limited to, alkylated carbohydrates (e.g., one or more hydroxyl groups that are methylated, ethylated, acetylated, or benzoylated), thiol carbohydrate (where one or more hydroxyl groups are replaced with —SH moiety), deoxy carbohydrates (where one or more —OH groups of the carbohydrate are replaced with —H), etc. More specifically, when referring to a carbohydrate, the term “derivative thereof” refers to a derivative of a carbohydrate in which one or more of the hydroxyl groups is replaced with hydrogen (e.g., 2-deoxy glucose, 5-deoxyglucose, etc.), an amine (e.g., amino sugars), a thiol (—SH) or a halogen, such as chloro, fluoro or iodo, (e.g., 5-fluoroglucose, 2-fluoroglucose, 5-chrologlucose, 2-chloroglucose, etc.). In addition, each of the monosaccharides can be an (L)-isomer or a (D)-isomer. The term “a thiol derivative” of a sugar refers to a sugar moiety in which the hydroxyl group that links the “B” moiety in the compound of Formula I is replaced with a sulfur atom. (i.e., the linkage between A and B moieties in compound of Formula I is sulfur). Similarly, the term “an amine or amino derivative” of a sugar refers to a sugar moiety in which the hydroxyl group that links the “B” moiety in the compound of Formula I is replaced with a nitrogen atom (i.e., the linkage between A and B is achieved by —NH— moiety).

The term “sugar” and “carbohydrate” may be used interchangeably herein and generally refers to a mono- or disaccharide or mixtures thereof. Exemplary carbohydrates that can be used in methods of the invention include but are not limited to, the following carbohydrates:

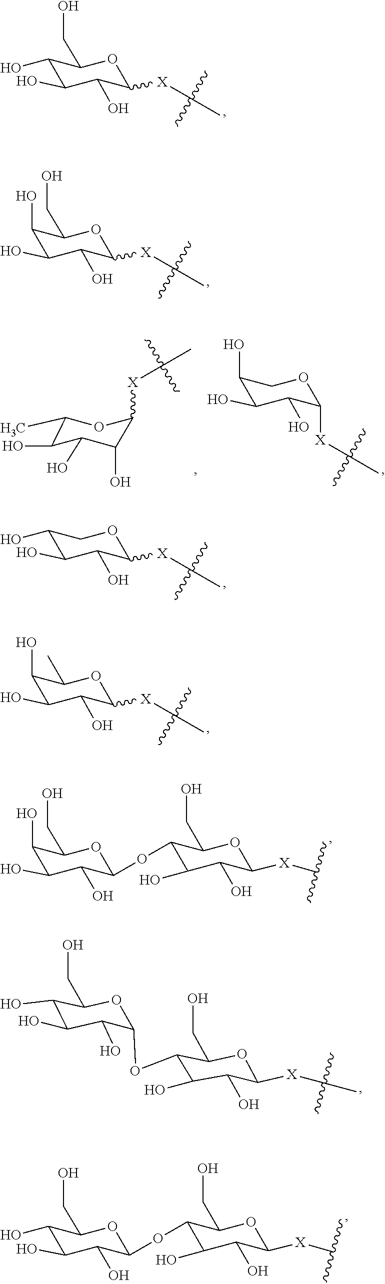

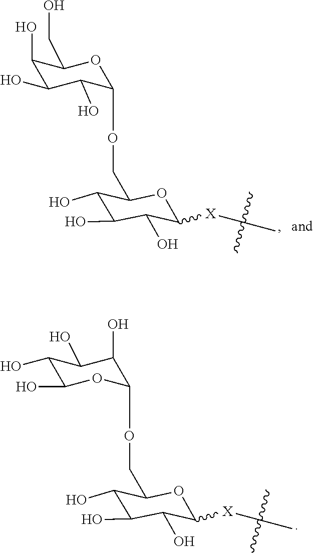

where X is O or S, and where one or more —OH is replaced with H, halogen, or —OR, where R is C₁₋₆ alkyl.

The term “monosaccharide” refers to any type of hexose of the formula C6H12O6 or a derivative thereof. The ring structure (i.e., ring type) of the monosaccharide can be a pyranose or a furanose. In addition, the monosaccharides can be an α- or β-anomer. Monosaccharide can be a ketonic monosaccharide (i.e., ketose), an aldehyde monosaccharide (i.e., aldose), or any type of hexose of the formula C₆H₁₂O₆ or a derivative thereof. Exemplary monosaccharides of the invention include, but are not limited to, allose, altrose, arabinose, fructose, galactose, glucose, gulose, idose, lxyose, psicose, rhamnose, ribose, ribulose, sorbose, tagatose, talose, xylose, xylulose, and derivative thereof. Each monosaccharide can also be independently an (L)-isomer or a (D)-isomer.

The term “disaccharide” refers to a carbohydrate composed of two monosaccharides. It is formed when two monosaccharides are covalently linked to form a dimer. The linkage can be a (1→4) bond, a (1→6) bond, a (1→2) bond, a (1→3) bond, etc. between the two monosaccharides. In addition, each of the monosaccharides can be independently an α- or β-anomer. Exemplary disaccharides that can be used in the present invention include, but are not limited to, cellobiose, chitobiose, dirhamnose, gentiobiose, isomaltose, isomaltulose, lactose, lactulose, laminaribose, leucrose, maltose, maltulose, melibiose, nigerose, sophorose, sucrose, trehalose, turanose, xylobiose, etc. Each of the monosaccharides can independently be a ketonic monosaccharide (i.e., ketose), an aldehyde monosaccharide (i.e., aldose), or any type of hexose of the formula C₆H₁₂O₆ or a derivative thereof. Each monosaccharide can also be independently an (L)-isomer or a (D)-isomer.

The terms “frother” and “frothing agent” refer to a C₁-C₆ alcohol (including CH₃OH, CH₃CH₂OH, CH₃CH₂CH₂OH, or (CH₃)₂CHOH) an agent that is active in froth flotation through its ability to change the surface tension of a liquid.

Compounds of Formula I can be readily prepared using, for example, procedures disclosed in commonly assigned U.S. patent application Ser. No. 15/358,159, which is incorporated herein by reference in its entirety.

For the sake of clarity and brevity, methods of the invention will now be described in removing uranium from uranium contaminated groundwater. However, it should be appreciated that the scope of the invention includes removing any metal ions, in particular, transition metal ions as well as rare earth metal ions from any sample, such as water, industrial waste, soil, etc.

Surprisingly and unexpectedly, the present inventors have discovered green biosurfactant rhamnolipid binds to uranium ions and acts as an effective collector in ion flotation. In one particular study by the present inventors, uranium-contaminated groundwater (˜440 μg L−1 U) from the Monument Valley processing site in northeast Arizona was used to determine the uranium removal efficacy of ion flotation with biosynthetic (bio-mRL) and three synthetic monorhamnolipids with varying hydrophobic chain lengths: Rha-C10-C10, Rha-C12-C12, and Rha-C14-C14. At the groundwater's native pH 8, and at an adjusted pH 7, no uranium removal from solution was observed by any collector. However, at pH 6.5 bio-mRL and Rha-C10-C10 removed 239.2 μg L−1 and 242.5 μg L−1 of uranium, respectively. By further decreasing the pH to 5.5, bio-mRL was able to reduce the uranium concentration to near or below the Environmental Protection Agency maximum contaminant level of 30 μg L−1. For the Rha-C12-C12 and Rha-C14-C14 collector ligands, decreasing the pH to 7 or below reduced the foam stability and quantity, such that these collectors were not suitable for treating this groundwater. To contextualize the results, a geochemical analysis of the groundwater was conducted, and a consideration of uranium speciation is described. Based on this study, the efficacy of monorhamnolipid-based ion flotation in real world groundwater has been demonstrated with suitable solution conditions and collectors identified.

Some of the advantages of methods of the invention include, but are not limited to, (i) glycolipid-based ion flotation can remediate uranium-contaminated groundwater; (ii) biosynthetic and synthetic rhamnolipids resulted in a similar amount of uranium ion removal; (iii) rhamnolipid-based ion flotation decreased uranium to the EPA max contaminant level; and (iv) uranium removal by ion flotation using glycolipid was pH dependent.

It should be appreciated that while the present invention is illustrated using rhamnose, as discussed above, rhamnose can be substituted or replaced with galactose, glucose, xylose, glucose, fructose, or any other mono- or disaccharides known to one skilled in the art. In fact, any glycolipids disclosed in a commonly assigned U.S. patent application Ser. No. 15/358,159 can be used in methods of the present invention. The glycolipid structure's fatty-acid tails (i.e., the “B” moiety) can be controlled to reduce the number of tails from 2 to 1 (i.e., R¹ is H or C₁-C₂₀ hydrocarbon optionally having 1, 2, or 3 carbon-carbon double bonds) and/or vary the tail length (i.e., number of carbon atoms in R¹ and/or m and/or n) and saturation. The demonstrated metal affinity of these glycolipids combined with the ability to change the sugar (i.e., moiety A) and tail structure (i.e., moiety B)—and thus modify the surfactant characteristics of the glycolipids (e.g., foaming)—means these glycolipids should exhibit similar and potentially better flotation performance for the recovery of uranium and other metals via the ion flotation application.

Representative glycolipids according to the invention include but are not limited to, the following compounds (in the “Generalized” structures, values of m and n will be understood to have values as indicated in the specific structures):

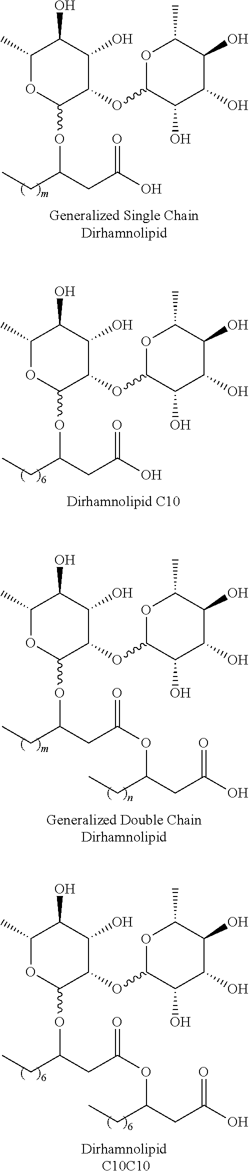

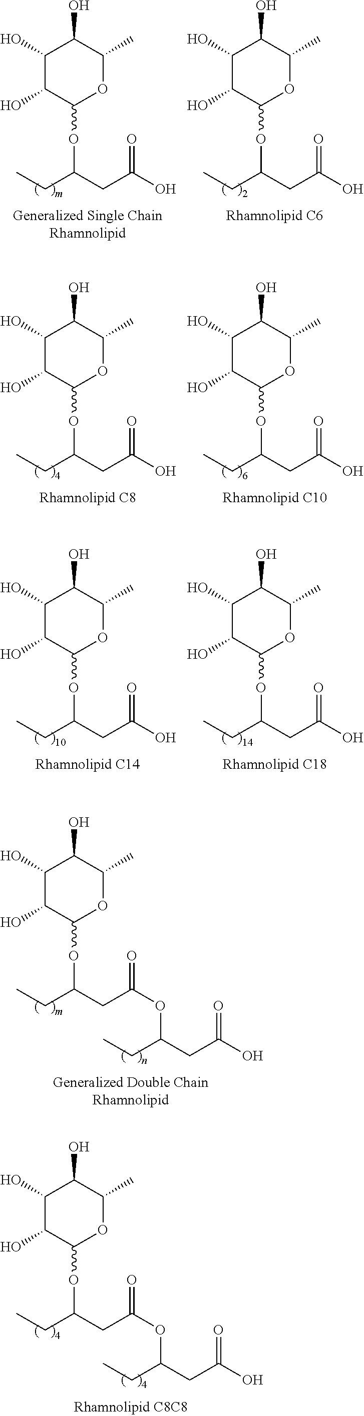

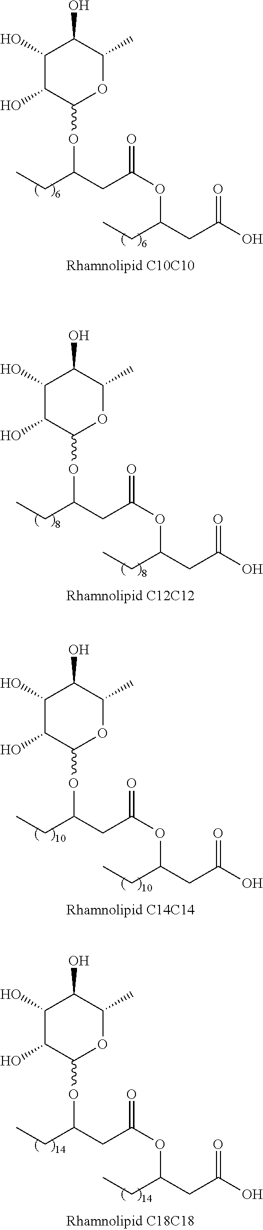

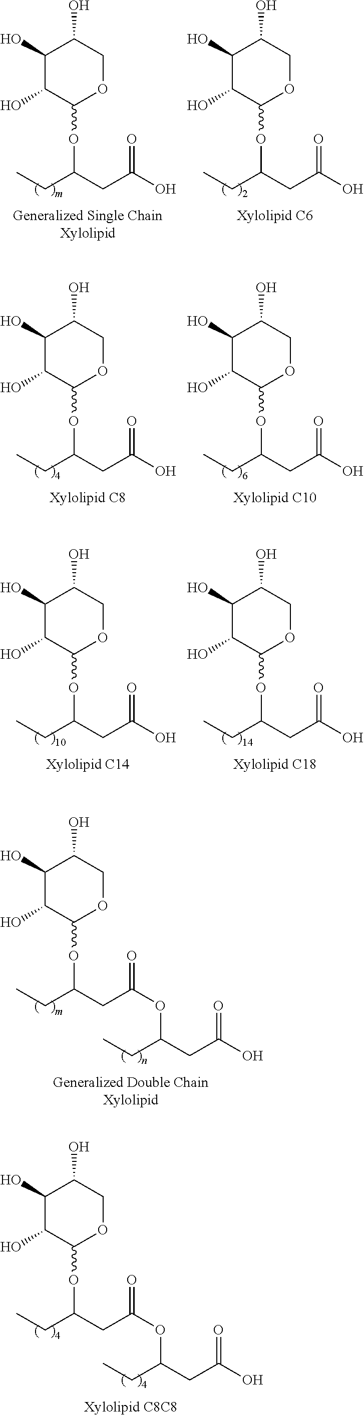

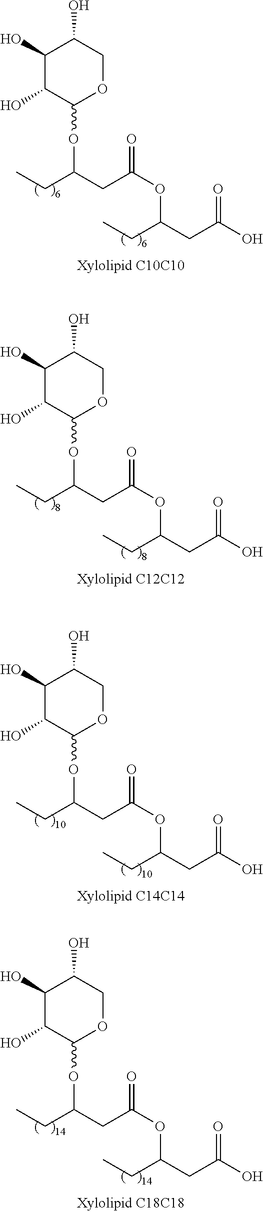

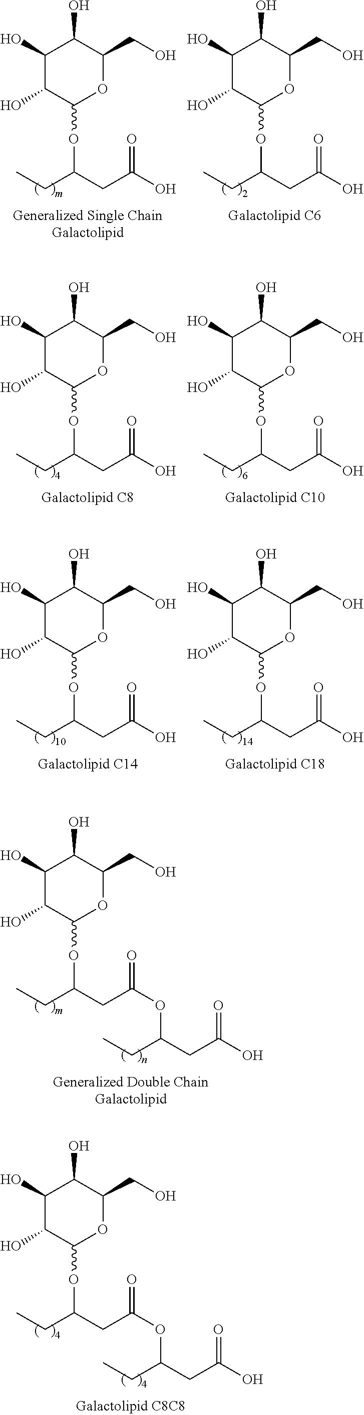

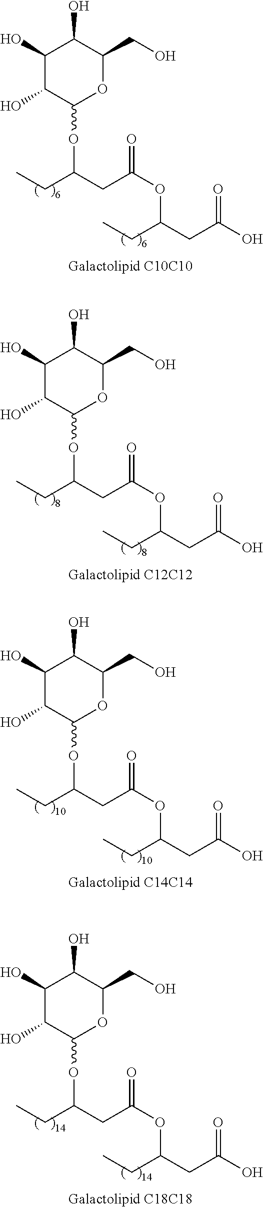

Exemplary metals that can be separated from a sample include but are not limited to, rare earth metals (such as Y, Ce, Pr, Nd, Pm, Sm, Eu, Gd, Tb, Dy, Ho, Er, Tm, Yb, Lu, La), valuable metals (such as Cu, Ag, Au, Pd, Pt), and metals of environmental concern (such as Pb, Cd, Zn, Tl, Hg).

Methods of the invention include combining, admixing, or contacting an aqueous solution of a glycolipid with a sample under conditions sufficient to form glycolipid-metal ion complex. The resulting glycolipid-metal ion complex is then separated from the mixture by any of the separation methods known to one skilled in the art, such as, but not limited to, flotation (such as ion flotation or precipitate flotation), precipitation, centrifugation, filtration, settling (e.g., gravitational settling), or combinations thereof.

Some of the factors that may affect formation of glycolipid-metal ion complex include, but are not limited to, temperature of the solution, concentration of the glycolipid in the aqueous solution, the nature of the glycolipid used, reaction time, rate of agitation or stirring of the mixture, pH of the glycolipid aqueous solution used, pH adjusting agent (if any), etc.

In some embodiments, e.g., for recovery of metals from acid mine drainage, the pH of the aqueous glycolipid solution or the mixture of the glycolipid solution and the sample is adjusted from pH of about 1 to pH of about 7, typically from pH of about 2 to pH of about 7, and often from pH of about 3 to pH of about 7. However, it should be appreciated that the scope of the present invention is not limited to these particular pH's. The actual pH can vary depending on a variety of factors including, but not limited to, the type of sample to be purified (e.g., groundwater, acid mine drainage, fracking water, etc.), nature of the glycolipid used, reaction time desired, temperature of glycolipid-metal ion complex formation used, concentration of glycolipid aqueous solution, etc. When referring to a numerical value, the terms “about” and “approximately” are used interchangeably herein and refer to being within an acceptable error range for the particular value as determined by one of ordinary skill in the art. Such a value determination will depend at least in part on how the value is measured or determined, e.g., the limitations of the measurement system, i.e., the degree of precision required for a particular purpose. For example, the term “about” can mean within 1 or more than 1 standard deviation, per the practice in the art. Alternatively, the term “about” when referring to a numerical value can mean ±20%, typically ±10%, often ±5% and more often ±1% of the numerical value. In general, however, where particular values are described in the application and claims, unless otherwise stated, the term “about” means within an acceptable error range for the particular value, typically within one standard deviation.

The amount of glycolipid used can vary depending on a variety of factors including, but not limited to, the amount of contamination of metal in the sample, the desired rate of glycolipid-metal ion complex formation, amount of metal ion to be removed, temperature of the reaction, etc. Typically, the ratio of the amount of glycolipid used to the amount of metal present in the sample ranges from about 200:1, typically from about 2:1. Where precipitate flotation or ion flotation is used, then the ratio of amount of glycolipid used to the amount of metal present in the sample ranges about 200:1 to about 10:1, or about 2:1, and often from about 150:1 to about 50:1 or about 10:1, for precipitate flotation or ion flotation. Where precipitation is used for separation, then the ratio of the amount of glycolipid used to the amount of metal present in the sample ranges may be about 2:1 to about 10:1, or about 2:1 to about 15:1.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLE

The following is a non-limiting example of the present invention. It is to be understood that said example is not intended to limit the present invention in any way. Equivalents or substitutes are within the scope of the present invention.

### Example 1: Ion Flotation

### Materials and Methods

Groundwater Collection and Analysis: The Monument Valley site is in Cane Valley, AZ, which is in northeastern Arizona. Groundwater was collected from monitoring well 662, which is down gradient of the source zone within the boundaries of the uranium groundwater plume. Samples were collected using dedicated bladder pumps and a QED Micropurge Controller. Groundwater was collected into a 50 L LDPE carboy (Thermo Scientific Nalgene) after the groundwater's pH and dissolved oxygen levels from the pump stabilized. The solution was stored as received, on the lab bench at ambient temperature until use.

For cation and anion analysis, groundwater was filtered (0.45 μM) then diluted with 2% trace metals grade nitric acid (Fisher Scientific, Waltham, MA) in metal-free polypropylene 15 mL centrifuge tubes (VWR, Radnor, PA). Cations were analyzed using an Elan DRC-II inductively coupled plasma mass spectrometer (ICP-MS). Anions (F⁻¹, Cl⁻¹, Br⁻¹, NO₂⁻¹, NO₃⁻¹, PO₄⁻¹, and SO₄⁻²) were analyzed by using a Thermo Scientific Dionex ICS-6000 high-performance ion chromatography system. Data generated in this geochemical analysis were used to model uranium speciation using Visual MINTEQ 3.1. Only major solution constituents—those greater than 10 μg L⁻¹ for metals or 5 μmol L⁻¹ for anions—were included in the experiment, and carbonates were excluded because they are stripped from solution when nitrogen is sparged in the flotation process.

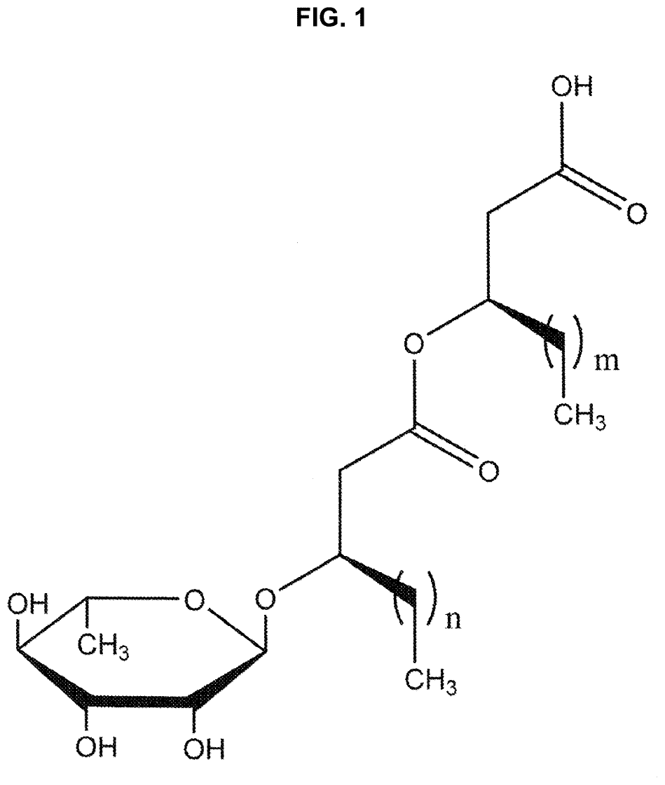

Chemicals: Both biosynthetic and synthetic monorhamnolipid collectors were tested for flotation efficiency. Structures of the monorhamnolipids utilized in this study are shown in FIG. 1. Biosynthetic monorhamnolipid tail lengths ‘m’ and ‘n’ range from 4 to 12 due to natural congener variation. Synthetic monorhamnolipids (Rha-C10-C10, Rha-C12-C12, or Rha-C14-C14) were synthesized with equal length tails where ‘m’=‘n’=6-14. The production and purification of biosynthetic monorhamnolipids (bio-mRL) from Pseudomonas aeruginosa ATCC 9027 has been previously described by Hogan et al., J. Hazard. Mater. 2017, 340, pp. 171-178. This organism exclusively produces monorhamnolipids—rhamnolipids with a single rhamnose sugar in the hydrophilic moiety of the molecule—as a congener mixture with varying hydrophobic tail lengths, in which the rhamnosyl-β-hydroxydecanoyl-β-hydroxydecanoate (C10-C10) dominates at 75-85 wt %. A molecular weight of 504 g mol⁻¹ (based on the C10-C10 structure) was used to estimate the bio-mRL concentration. Synthetic monorhamnolipids with decyl (Rha-C10-C10), dodecyl (Rha-C12-C12), and tetradecyl (Rha-C14-C14) hydrophobic moieties were prepared for comparative experiment. The molecular weights of the synthetic monorhamnolipids are 504.7, 560.8, and 616.9, respectively. All solutions were made using nanopure water (≥18 MΩ-cm). pH was adjusted with HNO₃ and NaOH. 100% molecular-grade ethanol was used as a frothing agent.

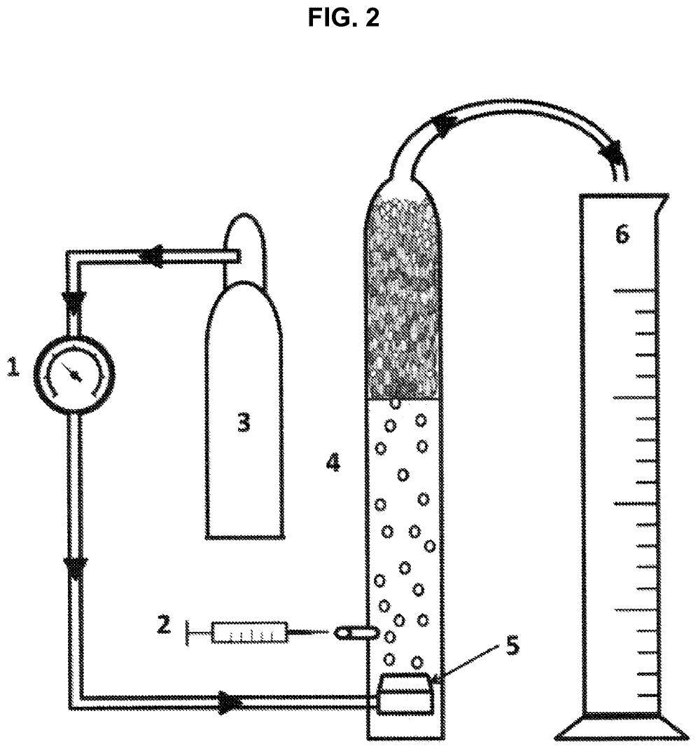

Flotation Apparatus: The flotation columns used in this study is shown in FIG. 2, including: rotameter 1; sampling syringe and port 2; compressed air cylinder 3; flotation column 4; gas dispersion frit 5; and foam collection reservoir 6 (FIG. 2 is reproduced from Hogan et al., Colloids and Interfaces, 2018, 2, p. 43). Each column was 50 cm tall with a diameter of 5.5 cm. Bubbles were generated using a glass frit (10-15 μm pore size) fused to the bottom of the column. A sampling port sealed with a rubber septum was located 2 cm above the frit. Foam was removed from the column using a glass funnel that collected foam into graduated cylinder. A gas manifold with three outlets enabled the operation of three columns concurrently, but each column was equipped with an independent rotameter to control gas flow rate.

Ion Flotation Experiments: The solution volume in each column was 250 mL with 250 μM monorhamnolipid collector and 0.5% (v/v) ethanol frother. Bubbles were produced using nitrogen gas at a flow rate of 50 mL min⁻¹. Experiments (data not shown) tested the effect of air versus nitrogen gas on the flotation of uranium; no differences were observed. However, nitrogen was used to reduce potential effects of CO₂ and carbonates in the system. Initial experiments were conducted at the native pH of the Monument Valley groundwater (pH 8.0), and subsequent experiments were conducted at a series of controlled pH values ranging from pH 7 to pH 5.5. Flotation solutions were mixed and used immediately. After the solution was added to the column, an initial column solution sample was collected, and then gas sparging was initiated. Columns were aerated until foam was no longer emitted from the column, typically about 45 min for columns with successful foam generation. When foam production ceased, the nitrogen flow was stopped, the column was manually swirled to evenly mix the solution and collapse remnant foam, and a final column solution sample was collected. Column samples were collected from the sampling port using a needle and syringe. For each sample, one mL of solution was collected then diluted for ICP-MS analysis using 2% trace metals grade nitric acid in metal-free polypropylene 15 mL centrifuge tubes. The volume of solution transported out of the column in the foam was measured gravitationally. The results are discussed both in terms of the uranium concentration in solution and the recovery percentage, which is calculated using Equation 1:

Recovery Percentage=[1−Cₜ/Cₒ]*100 (1)

where Cₒ is the initial uranium concentration in the column solution, and C_{f} is the final concentration.

### Results and Discussion

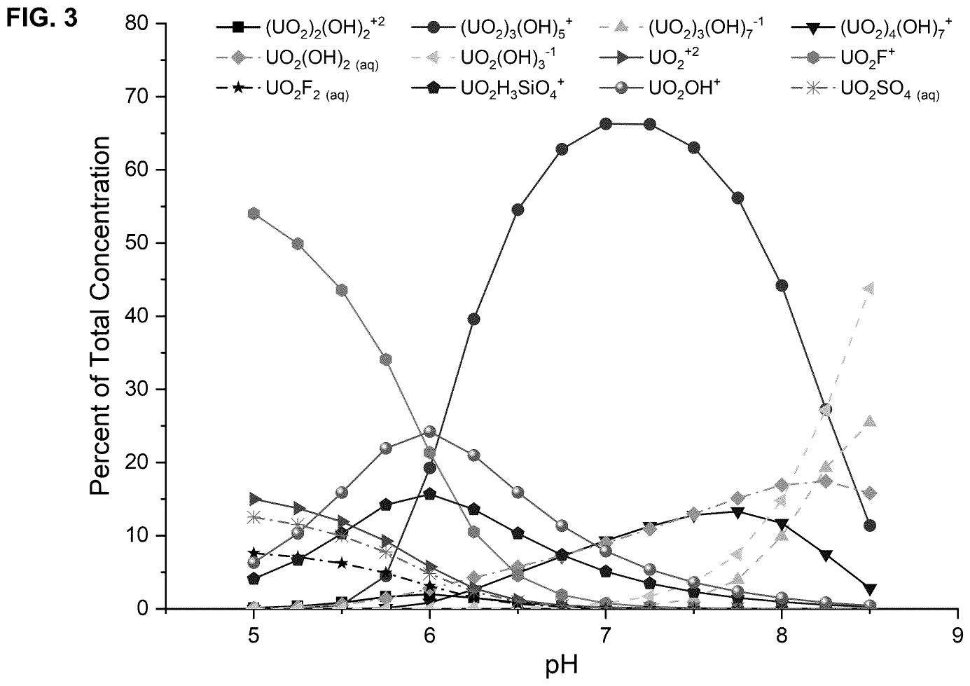

Monument Valley groundwater characteristics: A geochemical analysis of major metal (>10 μg L⁻¹) and anion (>5 μmol L⁻¹) constituents of the Monument Valley groundwater samples (Table 1) showed the largest metal(loid) constituents were Ca, K, Mg, Na, and Si, each in the mg L⁻¹ range. Uranium was present at 442.4 μg L⁻¹. The major anions measured above the detection limit of 5 μmol L⁻¹ were sulfate, nitrate, chlorine, and fluorine. Visual MINTEQ 3.1 modeling of uranium speciation (FIG. 3) shows the dependence of uranium speciation on pH in this system. At the native pH of the groundwater (pH 8.0), the predominant uranium species is predicted to be cationic (UO₂)₃(OH)₅⁺¹ accounting for 44.2% of the total concentration along with 16.9% UO₂(OH)_{2(aq)}, 14.8% UO₂(OH)₃⁻¹, 11.7% (UO₂)₄(OH)₇⁺¹, 9.9% (UO₂)₃(OH)₇⁻¹, and 2.4% of other cationic species. Thus, at pH 8, the total percentage of cations is 58.3%. As pH in the system is decreased to pH 5.5, modeling indicates an increase in the concentration of uranium cations, such that the total percentage of cations is 83.1%: 43.6% UO₂F⁺¹_{(aq)}, 15.9% UO₂OH⁺¹, 11.9% UO₂⁺², 10.3% UO₂H₃SiO₄₊₁, and less than 1% each of (UO₂)(OH)₂⁺² and (UO₂)₃(OH)₅⁺. This observation is consistent with other uranium speciation modeling results.

TABLE 1
Major metal (>10 μg L⁻¹) and anion (>5 μmol L⁻¹)
constituents present in Monument Valley groundwater.

| Constituent | μg L⁻¹ | Constituent | μg L⁻¹ |
|---|---|---|---|
| Al | 44.8 | Si | 7,070 |
| B | 56.0 | Sr | 516 |
| Ba | 38.2 | U | 442 |
| Ca | 52,200 | V | 28.5 |
| K | 1,800 | Cl | 270 |
| Li | 34.8 | F | 45.1 |
| Mg | 36,000 | NO₃ | 326 |
| Na | 26,500 | SO₄ | 1580 |

Effect of pH on performance of biosynthetic rhamnolipid in ion flotation: An initial control experiment was performed to test whether the bio-mRL could float uranium from Monument Valley groundwater without pH modification. Since uranium speciation modeling suggests a concentration of 58.3% cationic species in the system at pH 8 (FIG. 3), it was believed that ion flotation would be effective at this pH without requiring solution amendment. Results did not support this belief, showing that despite copious foam and typical column operation, flotation did not remove any significant amount of uranium from solution (data not shown).

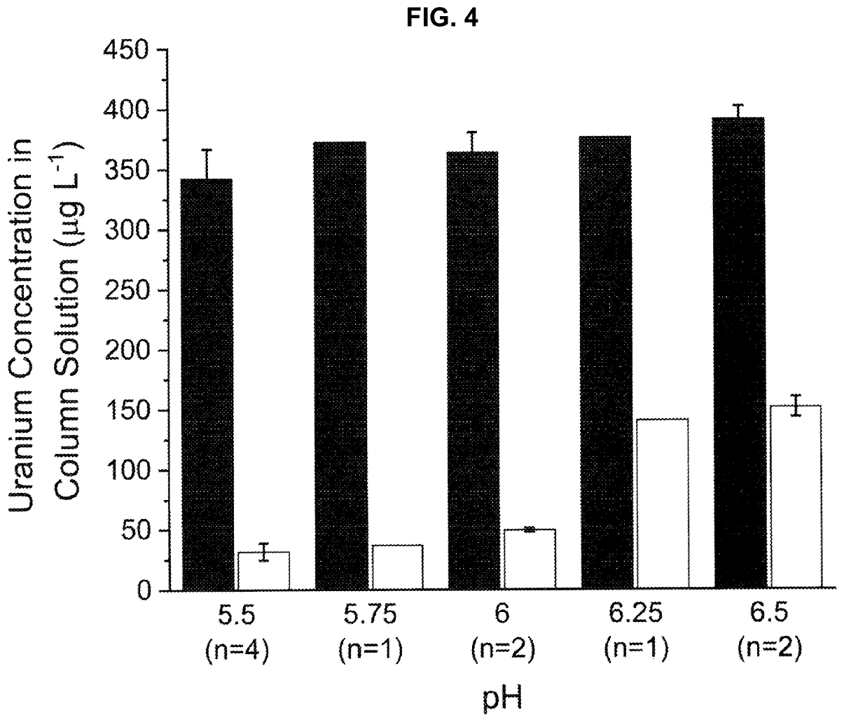

A second set of experiments was performed to test the impact of pH on flotation using the bio-mRL. At pH 6.5, the removal percentage was 64.4%. Though this was a substantial reduction in uranium concentration, the final concentration remained above the maximum contaminant level (MCL) of 30 μg L⁻¹ set by the U.S. Environmental Protection Agency (EPA) for community water systems. Using this MCL as a target, additional experiments were conducted wherein pH was further decreased in 0.25 pH unit increments from pH 6.5 to pH 5.5, using samples with the initial concentrations indicated by the black bars in FIG. 4. In each of the tested conditions, bio-mRL successfully supported the generation and collection of foam, despite approaching the monorhamnolipid pKₐ (5.5). Increasing uranium removal was observed as the pH was decreased, and the maximum removal was achieved at the lowest tested pH (5.5) with a mean of 31.7 μg L⁻¹ (92.6% removed) and a range of 19.9 to 38.0 μg L⁻¹ (see FIG. 4, white bars). The average volume of solution removed from the column and recovered with the foam at pH 5.5 was 15.0±3.7 mL (6% of the original volume)—this volume was characteristic for the apparatus and experimental conditions, though volume transfers as high as 25.9 mL (10.4%) were observed in other treatments. Thus, at pH 5.5 rhamnolipid-based flotation was able to remediate 94% of the initial water volume to near or below the EPA MCL.

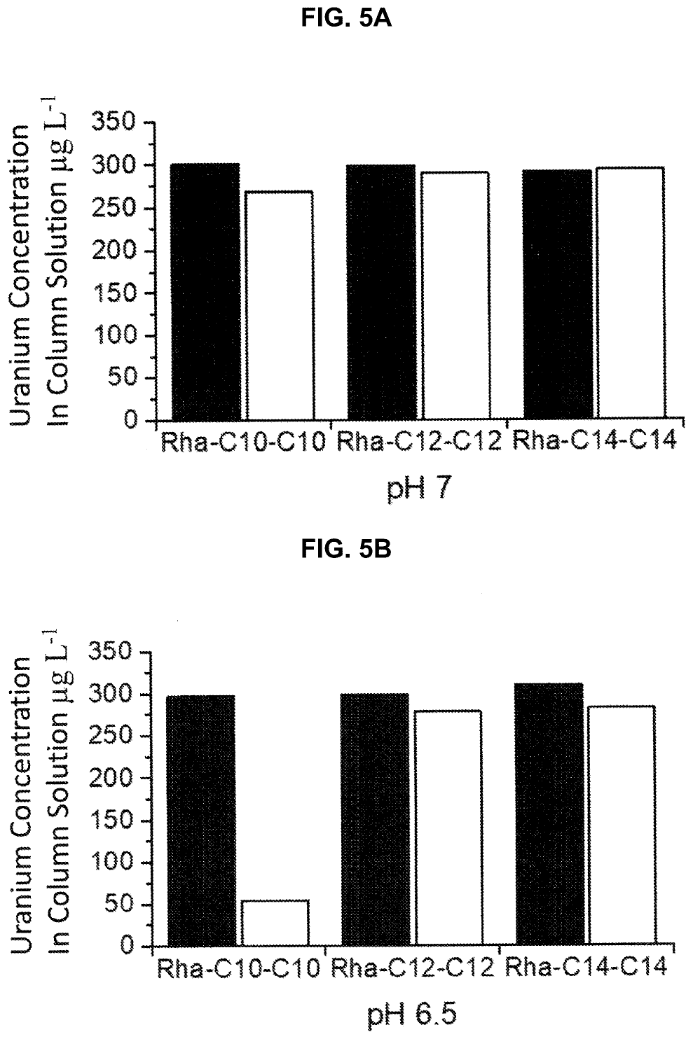

Effect of pH on performance of synthetic rhamnolipids in ion flotation: A third set of experiments was conducted to evaluate the performance of the three synthetic monorhamnolipids: Rha-C10-C10, Rha-C12-C12, and Rha-C14-C14. As described for the bio-mRL, flotation was not successful with any of these synthetic rhamnolipids at the natural Monument Valley pH of 8 (data not shown). At pH 7, foaming and normal flotation behavior was observed for Rha-C10-C10, but very little uranium was removed (FIG. 5A: black bars indicate initial sample concentration; white bars indicate final concentrations). No significant amount of uranium was removed by either the Rha-C12-C12 (which sporadically produced small amounts of foam) or by the Rha-C14-C14 (which did not produce a foam at all). Reduction of the pH to 6.5 resulted in substantial 81.9% uranium removal using the Rha-C10-C10 collector, while removals for Rha-C12-C12 (6.6%) and Rha-C14-C14 (8.7%) remained low (FIG. 5B: black bars indicate initial sample concentration; white bars indicate final concentrations).

A performance comparison of the bio-mRL and the Rha-C10-C10 at pH 6.5 shows that the bio-mRL is equally effective as the Rha-C10-C10. Bio-mRL reduced the uranium concentration from 390.5 to 151.3 μg L⁻¹, and Rha-C10-C10 reduced the concentration from 296.1 to 53.7 μg L⁻¹. These numbers reflect nearly equivalent reductions of 239.2 μg L⁻¹ for bio-mRL and 242.4 μg L⁻¹ for Rha-C10-C10 (FIGS. 4 and 5B, respectively).

Examination of Rha-C12-C12 and Rha-C14-C14 behavior suggests that they are not well suited for ion flotation of uranium (in this system). At pH 8 both of these molecules formed copious amounts of stable, collectable foam, but they did not collect metals. On the other hand, at pH 7 and pH 6.5, both collectors failed to form stable foams that could be collected from the column. Without being bound by any theory, it is believed that the failure to form a stable foam is likely due to decreased solubility of the longer chain molecules, especially as the pH approaches the pKₐ of 5.5 identified for biosynthetic monorhamnolipids. Thus, since no foam was removed from the columns during the experiment, any reduction in solution metal concentration can be attributed to the formation of monorhamnolipid/metal precipitates. These precipitates either accumulate at the solution surface or adhere to the column surfaces, as was conspicuously observed in different solution conditions. It is worth noting that decreasing pH also had an observable effect on the foam formation for the bio-mRL and Rha-C10-C10 collectors. While foaming was still substantial, visual examination of the foam produced at lower pH showed an increase in coarseness and a decrease in foam abundance as pH was decreased, presumably due to decreasing solubility.

The ability of bio-mRL and Rha-C10-C10 to remain functional to at least pH 5.5 indicates at least the following: (i) this remediation application is a viable option for moderately acidic solutions; and (ii) when selecting a collector, chain length is an important parameter to consider. In this system, the decyl chains (bio-mRL and Rha-C10-C10) enabled effective removal of uranium while dodecyl and tetradecyl were not as effective. Synthesis of shorter chained molecules may prove even more effective in some instances, so long as the efficacy and solubility are balanced against the requisite collector surface activity for a particular metal ion. In other systems, increasing the chain length of the collector molecule was shown to decrease the final solution concentration of the target metal and decrease the time for this minimum concentration to be reached. Thus, the longer chain monorhamnolipids should still be considered as collectors for applications where the pH (i.e., monorhamnolipid solubility) and/or target metal (i.e., metal speciation) are amenable for ion flotation.

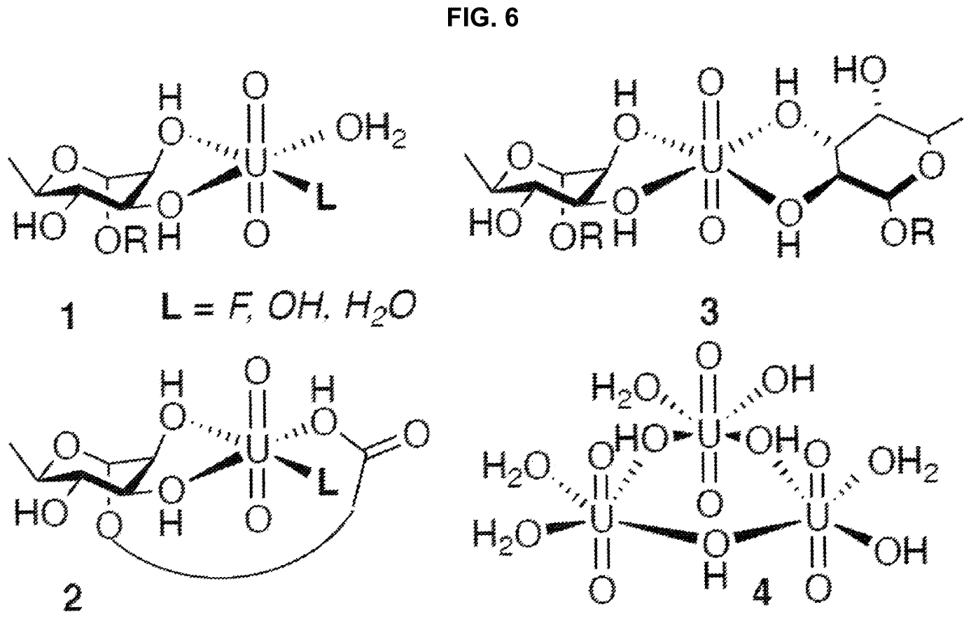

Effect of Uranium Speciation on Rhamnolipid Performance: Again without being bound by any theory, it is believed that the failure of rhamnolipids to remove a significant amount of uranium above pH 6.5 suggests that there is a uranium speciation issue interfering with the monorhamnolipids' binding activity. While the specific coordination mode of monorhamnolipid with Pb has been proposed by others, the coordination to metal centers is not well-characterized, in particular with uranium oxo species. Metal-carbohydrate complexes, however, are well-characterized. Although the coordination is not specifically known, uranium oxo complexes and ligand coordination has been well-studied, and the proposed structures do provide some insight into the U-species dependent coordination of monorhamnolipids. As shown in FIG. 3, the predominant uranium species at pH values where flotation was successful (pH 5.5-6.5) are UO₂(OH)⁺¹, UO₂F⁺¹, UO₂H₃SiO₄⁺¹, and UO₂⁺¹ (UO₂⁺¹ is most likely a fully hydrated species). The structures of UO₂(OH)⁺¹, UO₂F⁺¹, and UO₂⁺¹ are probably similar with octahedral coordination geometry. The protonated uranium oxo species UO₂H₃SiO₄⁺¹ will also fit this geometry and should be effectively bound by monorhamnolipid. In FIG. 6, shows four potential binding modes (structures 1 to 4) of monorhamnolipid with these uranyl cations. The plausible coordination modes are monorhamnose-uranyl species coordinated through the 3 and 4-hydroxy moieties of rhamnose (FIG. 6, structure 1), a trichelated species (FIG. 6, structure 2), and the dilipid-bound species (FIG. 6, structure 3) that is likely only probable for UO₂⁺. Because interactions of water are relatively weak and labile, the proposed species are likely due to the stronger interactions from the chelation effect of the sugar moiety and the carboxylic acid. At higher pH, increased availability of hydroxide leads to the formation of the trinuclear species (UO₂)₃(OH)₅⁺¹ (FIG. 6, structure 4) as modeled in FIG. 3. It is believed that strong interactions do not exist between structural species 4 with monorhamnolipid for the reasons discussed below.

Despite their cationic speciation, the predominance of the relatively large and polyatomic (UO₂)₃(OH)₅⁺¹ and (UO₂)₄(OH)₇⁺¹ species results in the poor flotation performance at pH 7 and pH 8. Monorhamnolipid-metal interaction strength has been correlated to the charge to ionic radius ratio. These large species have a small charge-to-ionic radius ratio weakening the monorhamnolipid-metal interaction and yielding poor flotation activity. Furthermore, the proposed monorhamnolipid binding mechanism is based on a binding pocket with a “bite” size defined by the molecular structure. The large size of the predominant species at higher pH levels is likely incompatible with monorhamnolipid's binding pocket. In addition, uranium-hydroxide coordination is very strong, excluding other ligands; by adding sufficient acid to the system, hydroxides protonate and their binding strength diminishes. Monorhamnolipid then becomes the predominant ligand in the system enabling effective flotation. These explanations are supported both by the data demonstrating flotation becomes increasingly effective and the modeling showing uranium speciation trends towards smaller species (FIG. 3) with decreasing pH. This analysis also highlights the importance of selecting uranium systems within (or amending systems to) an acidic regime when considering applications for ion flotation as a remediation strategy.

Parameters Affecting Rhamnolipid Performance in Ion Flotation: It is believed that the major drivers of flotation success are pH, target metal, and solution constituents. As the present invention shows, removal of uranium increases as pH decreases from pH 8 to pH 5.5. Opposite trends have been noticed by others when testing flotation of a rare earth element (REE) mixture containing La(III), Ce(III), Gd(III), and Yb(III). At pH 5.5 and pH 8, removal of these elements was poor (<20% recovery) but there was a dramatic increase to 90-95% recovery at pH 9. At pH 11, removal of La(III) remained high, but recovery of other metals dropped and a selectivity sequence of La(III)>Yb(III)>Gd(III)>Ce(III) was observed, suggesting potential for separation applications. The narrow pH range that enabled the best recovery has been attributed to at least two effects: pH-induced formation of metal/rhamnolipid complexes or the formation of (monorhamnolipid-repulsive) anionic REE complexes. It should be noted others on the other hand found an intermediate result. When recovering Cd(II), a nonlinear trend was observed between pH 4 and pH 10 with a maximum recovery at pH 6. The decreased Cd recovery in the acidic regime has been attributed to a change in rhamnolipid surface activity and charge (becoming uncharged); the decrease under alkaline conditions was attributed to the formation of metal hydrates or a decrease in rhamnolipid adsorption density due to the increase in ionic strength as pH regulator NaOH was added. Taken together, it is clear pH enforces a two-sided limit on efficacy in rhamnolipid-based ion flotation: on the acidic side of the scale, rhamnolipid solubility and activity controls success, while on the alkaline side, success is limited by metal speciation.

In addition to pH, the concentration of target and non-target metals in solution also controls flotation efficacy. For example, the flotation of REE and U from a metal-dense, acidic mining solution adjusted to pH 5.5 failed to form any significant foam (using bio-mRL). Subsequent experiments at near neutral pH (and thus lower metal concentrations due to pH-induced metal precipitation) successfully recovered REE and U even in the presence of competing cations at orders-of-magnitude higher concentrations. These results highlight the dependency of total solution chemistry on flotation. Indeed, even single elements can lead to variation in performance. For example, some have showed flotation of La(III), Cd(II), and Cs(I) (individually) at metal-to-monorhamnolipid ratios of 1:2, 1:5, and 1:10 was successful except for La at the 1:2 ratio where a precipitate scum formed instead of a collectable foam. This result was attributed to insufficient monomers in solution capable of supporting a foam. Others did not note any precipitate formation when floating Cd(II), but a reduction in Cd recovery was observed as the Cd concentration was increased. The reduction was attributed to a decrease in rhamnolipid on a mole per mole basis since relatively fewer rhamnolipid molecules were available to transport the increasing Cd concentrations. It is possible to overcome these mass limitations by increasing collector concentrations, but doing so may lead to detrimental effects such as increased water transport, lower enrichment, and decreased transport kinetics as micelles form and competition for space on the air-water interface increases. Increasing collector concentrations increases material costs for remediation applications as well.

Disaccharide glycolipids: Dirhamnolipid from the supplier Evonik was used as received, and a representative structure is shown below:

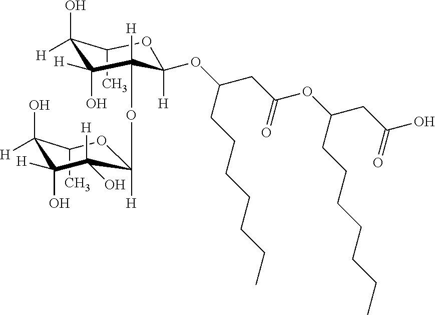

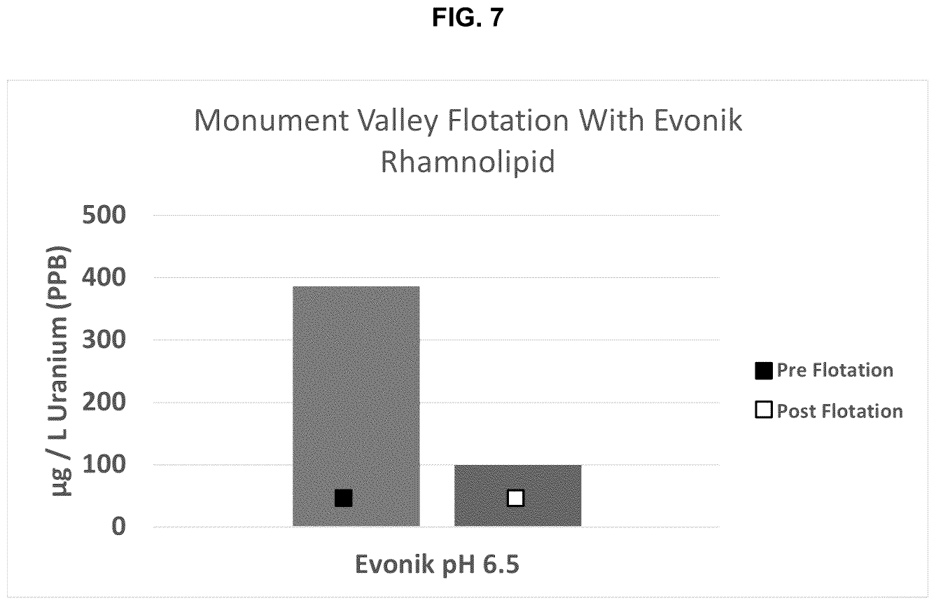

As described above in the removal of uranium from groundwater using monorhamnolipids and ion flotation, a similar process was used at pH 6.5 using the dirhamnolipid. The results are shown in FIG. 7.

As the data shows, the dirhamnolipid removed uranium with about 75% removal after a single treatment.

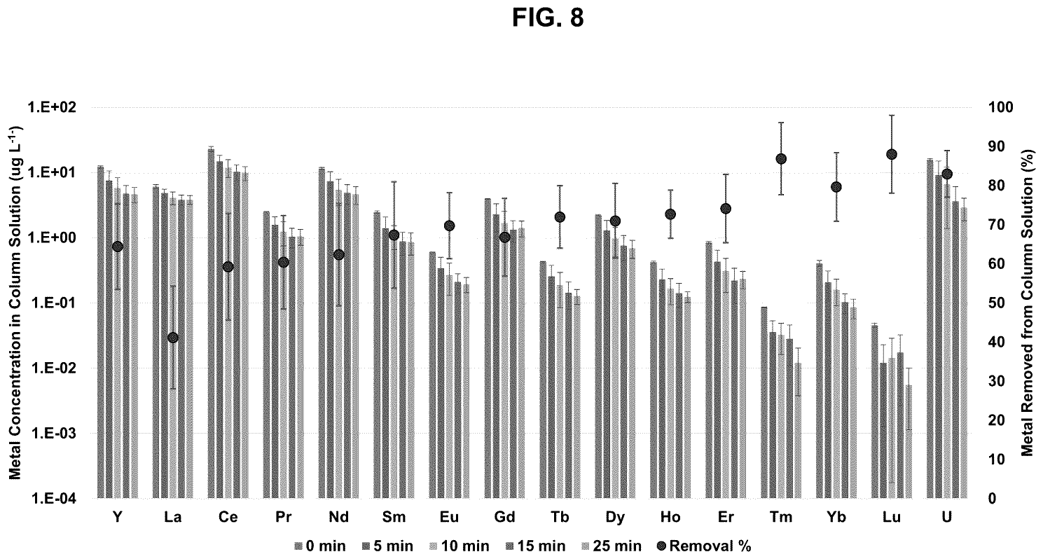

Ion Flotation of Rare Earth Elements Using Monorhamnolipid C10C10: Synthetic monorhamnolipid C10-C10 was used at pH 6.5 to recover rare earth metals from a real world mining raffinate solution. Solution metal concentrations were measured at 0, 5, 10, 15, and 25 min then at the cessation of foam formation. The results are shown in FIG. 8.

As the data shows, the removal efficiency of rare earth elements by monorhamnolipid C10C10 at pH 6.5 ranged from about 60 to 90% at the termination of the experiment (circles in FIG. 8).

### Example 2.1: Precipitation, Centrifugation, and Filtration

Structural variants of synthetic glycolipids were used to show the effect of the number and length of the hydrophobic tail(s) on rhamnolipid as a chemical precipitant. Table 2 shows just the rhamnolipid variants used. Solutions (10 mM) were made using nanopure water (≥18 MΩ-cm) then adjusted to a pH of 6.9±0.05 with HNO₃ and NaOH. These solutions were made in advance and stored in the cold room for up to one month. They were brought to room temperature before each experiment.

TABLE 2
The rhamnolipid structures (single chain and double chains) tested.

| | | | | |
|---|---|---|---|---|
| | | Molecular | Chemical | |
| Rhamnolipid | Abbreviation | Weight | Formula | Structure |

Single Chains

| | | | | |
|---|---|---|---|---|
| Rhamnolipid C6 | Rha-C6 | 278.3 | C₁₂H₂₂O₇ | 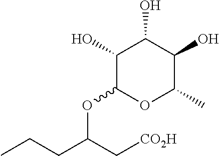 [SMILES: CCCC(CC(=O)O)OC1O[C@@H](C)[C@H](O)[C@@H](O)[C@H]1O] |
| Rhamnolipid C10 | Rha-C10 | 334.4 | C₁₆H₃₀O₇ | 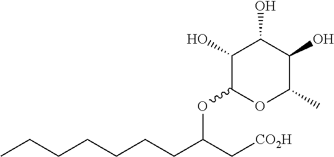 [SMILES: CCCCCCCC(CC(=O)O)OC1O[C@@H](C)[C@H](O)[C@@H](O)[C@H]1O] |
| Rhamnolipid C14 | Rha-C14 | 390.5 | C₂₀H₃₈O₇ | 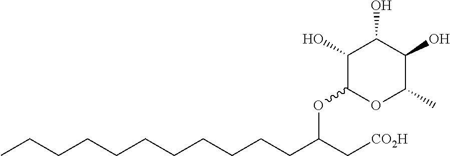 [SMILES: CCCCCCCCCCCC(CC(=O)O)OC1O[C@@H](C)[C@H](O)[C@@H](O)[C@H]1O] |
| Rhamnolipid C18 | Rha-C18 | 520.66 | C₂₄H₄₆O₇ | 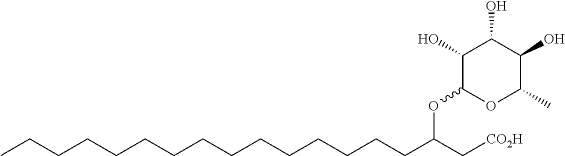 [SMILES: CCCCCCCCCCCCCCCC(CC(=O)O)OC1O[C@@H](C)[C@H](O)[C@@H](O)[C@H]1O] |

Double Chains

| | | | | |
|---|---|---|---|---|
| Rhamnolipid C10C10 | Rha-C10C10 | 504.7 | C₂₆H₄₈O₉ | 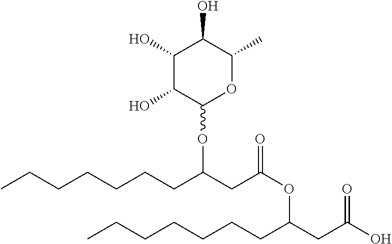 [SMILES: CCCCCCCC(CC(=O)O)OC(=O)CC(CCCCCCC)OC1O[C@@H](C)[C@H](O)[C@@H](O)[C@H]1O] |
| Rhamnolipid C12C12 | Rha-C12C12 | 560.8 | C₃₀H₅₆O₉ | 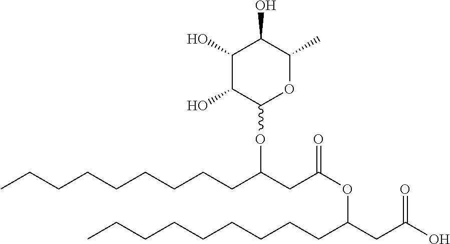 [SMILES: CCCCCCCCCC(CC(=O)O)OC(=O)CC(CCCCCCCCC)OC1O[C@@H](C)[C@H](O)[C@@H](O)[C@H]1O] |
| Rhamnolipid C14C14 | Rha-C14C14 | 616.9 | C₃₄H₆₄O₀ | 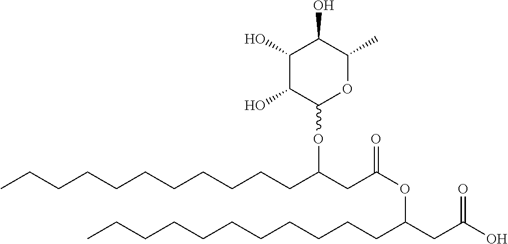 [SMILES: CCCCCCCCCCCC(CC(=O)O)OC(=O)CC(CCCCCCCCCCC)OC1O[C@@H](C)[C@H](O)[C@@H](O)[C@H]1O] |

Three metals were tested for binding with rhamnolipid. Lead as Pb(NO₃)₃ (Fisher Scientific, Waltham, MA, 99% purity), was chosen to represent a metal with high rhamnolipid-metal conditional stability constant; lanthanum as La(NO₃)₃ 6(H₂O) (Sigma-Aldrich, St. Louis, MO, 99.99% purity) was chosen as a representative REE; and magnesium as Mg(NO₃)₂ 6(H₂O) (Sigma-Aldrich, St. Louis, MO, 99% purity) was chosen to represent a metal with a low rhamnolipid-metal conditional stability constant (Hogan et al., 2017). Metal solutions (5 mM) were made using nanopure water (≥18 MΩ-cm) in 20 ml glass scintillation vials. All solutions were used within 15 min to ensure minimal adsorption to the glass vial.

Three glycolipid-based treatments were evaluated to remove metal from solution: (1) the addition of glycolipid with no further treatment (gravitational precipitation), (2) the addition of a glycolipid followed by filtration or (3) the addition of a glycolipid followed by centrifugation. Each metal (Pb, La, and Mg) was evaluated separately with each of the glycolipid structures. For each experiment, a metal solution was mixed with a glycolipid solution in a 1.7 ml reaction tube at varying molar ratios of glycolipid to metal: 0:1 (glycolipid-free control), 2:1, 5:1, and 10:1. The reactions were mixed in the order of nanopure water (≥18 MΩ-cm), glycolipid solution (10 mM), then metal solution (5 mM). The metal and glycolipid solutions were continuously stirred as they were aliquoted into the reaction tubes. The experiment was performed in triplicate.

The reaction tubes were mixed overnight (16 to 24 h) and then three samples were collected from each tube to measure the effect of each of the three treatments: a glycolipid only treatment sample, a filtration treatment sample, and a centrifugation treatment sample. Briefly, for the glycolipid only treatment samples all tubes were vortexed for approximately 5 sec immediately before removing 0.1 ml into a dilution tube of 2% trace metal grade nitric acid (12 to 14 ml) to minimize the settling of particles. For the filtration samples each reaction tube was vortexed for 5 sec and then a 0.6 ml aliquot was collected with a 1 ml syringe. A GHP acrodisc disk filter (hydrophilic polypropylene membrane, 0.45 μm pore size, 25 mm disc diameter) from Pall Life Sciences (Port Washington, New York) was then placed onto the end of the syringe and the solution was pushed through the filter. The void space in the disk filter holds approximately 0.5 ml, therefore around 0.1 ml of filtrate was collected into a dilution tube. For the centrifugation sample, the remaining solution was centrifuged at 10,000 RCF for 15 min. Approximately 0.1 ml of the supernatant was aliquoted into a dilution tube. Following collection, all samples were taken for metal analysis using ICP-MS.

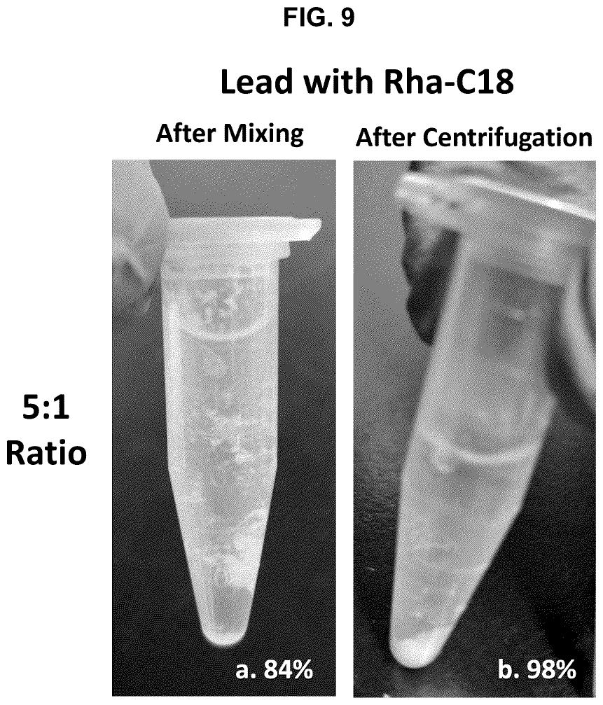

FIG. 9 shows the effect of glycolipid addition to a solution of La at a 5:1 ratio. After mixing alone (gravitational settling), 84% of the metal was precipitated from solution. An additional centrifugation step increased La removal to 98%. Full results for glycolipids after gravitational settling, centrifugation, and filtration are shown in Tables 3-5 below:

TABLE 3
Concentrations (mmol/L) of lead in the reaction tube initially and after the three
treatments. The molar ratio (column 2) is rhamnolipid:lead. The efficiency of
removal is presented as percent in parentheses below the concentrations.

| | | | | | Treatment 3: |
|---|---|---|---|---|---|
| | | | Treatment 1: | Treatment 2: | Rhamnolipid |
| | | | Rhamnolipid | Rhamnolipid | and |
| | | Initial | Only | and Filtration | Centrifugation |
| | Molar | Concentration | Cᵣ (mmol/L) | C_{f} (mmol/L) | C_{c} (mmol/L) |
| Rhamnolipid | Ratio | (mmol/L) | (Rᵣ) | (R_{f}) | (R_{c}) |
| Rha C6 | 2 to 1 | 0.321 ± 0.008 | 0.323 ± 0.005 | 0.115 ± 0.022 | 0.313 ± 0.003 |
| | | | (NR) | (64.0%) | (2.2%) |
| | 5 to 1 | | 0.329 ± 0.014 | 0.046 ± 0.017 | 0.322 ± 0.004 |
| | | | (NR) | (85.8%) | (NR) |
| | 10 to 1 | | 0.316 ± 0.008 | 0.119 ± 0.018 | 0.323 ± 0.005 |
| | | | (1.4%) | (62.7%) | (NR) |
| Rha C10 | 2 to 1 | 0.329 ± 0.003 | 0.320 ± 0.012 | 0.320 ± 0.048 | 0.320 ± 0.045 |
| | | | (2.7%) | (88.8%) | (2.5%) |
| | 5 to 1 | | 0.293 ± 0.006 | 0.028 ± 0.031 | 0.288 ± 0.002 |
| | | | (10.7%) | (91.6%) | (12.2%) |
| | 10 to 1 | | 0.232 ± 0.009 | 0.001 ± 0.000 | 0.231 ± 0.003 |
| | | | (29.3%) | (99.8%) | (29.7%) |
| Rha C14 | 2 to 1 | 0.321 ± 0.008 | 0.278 ± 0.011 | 0.031 ± 0.030 | 0.194 ± 0.006 |
| | | | (13.3%) | (90.2%) | (39.4%) |
| | 5 to 1 | | 0.315 ± 0.004 | 0.000 ± 0.000 | 0.315 ± 0.006 |
| | | | (1.6%) | (100.0%) | (1.8%) |
| | 10 to 1 | | 0.311 ± 0.008 | 0.001 ± 0.001 | 0.327 ± 0.001 |
| | | | (3.0%) | (99.7%) | (NR) |
| Rha C18 | 2 to 1 | 0.272 ± 0.010 | 0.123 ± 0.002 | 0.027 ± 0.001 | 0.132 ± 0.001 |
| | | | (54.8%) | (90.1%) | (51.6%) |
| | 5 to 1 | | 0.044 ± 0.008 | 0.000 ± 0.002 | 0.005 ± 0.002 |
| | | | (83.9%) | (99.9%) | (98.1%) |
| | 10 to 1 | | 0.255 ± 0.015 | 0.000 ± 0.001 | 0.014 ± 0.001 |
| | | | (6.2%) | (99.9%) | (94.7%) |
| Rha C10C10 | 2 to 1 | 0.272 ± 0.010 | 0.109 ± 0.006 | 0.017 ± 0.007 | 0.091 ± 0.003 |
| | | | (60.1%) | (93.8%) | (66.6%) |
| | 5 to 1 | | 0.271 ± 0.013 | 0.002 ± 0.002 | 0.005 ± 0.000 |
| | | | (0.5%) | (99.2%) | (98.1%) |
| | 10 to 1 | | 0.267 ± 0.005 | 0.083 ± 0.009 | 0.214 ± 0.004 |
| | | | (1.9%) | (69.7%) | (21.4%) |
| Rha C12C12 | 2 to 1 | 0.272 ± 0.010 | 0.211 ± 0.010 | 0.069 ±0.018 | 0.146 ± 0.008 |
| | | | (22.4%) | (74.8%) | (46.4%) |
| | 5 to 1 | | 0.204 ± 0.004 | 0.049 ± 0.021 | 0.047 ± 0.003 |
| | | | (25.0%) | (81.8%) | (82.8%) |
| | 10 to 1 | | 0.249 ± 0.10 | 0.059 ± 0.028 | 0.129 ± 0.002 |
| | | | (8.6%) | (78.3%) | (52.8%) |
| Rha C14C14 | 252% to 1 | 0.272 ± 0.010 | 0.213 ± 0.003 | 0.094 ± 0.010 | 0.1414 ± 0.002 |
| | | | (21.4%) | (65.6%) | (48.1%) |
| | 5 to 1 | | 0.243 ± 0.022 | 0.016 ± 0.001 | 0.042 ± 0.001 |
| | | | (10.8%) | (94.1%) | (84.4%) |
| | 10 to 1 | | 0.241 ± 0.011 | 0.005 ± 0.001 | 0.008 ± 0.000 |
| | | | (11.3%) | (98.2%) | (97.1%) |

TABLE 4
Concentrations (mmol/L) of lanthanum in the reaction tube initially and after the
three treatments. The molar ratio (column 2) is rhamnolipid:lanthanum. The efficiency
of removal is presented as percent in parentheses below the concentrations.

| | | | | | Treatment 3: |
|---|---|---|---|---|---|
| | | | Treatment 1: | Treatment 2: | Rhamnolipid |
| | | | Rhamnolipid | Rhamnolipid | and |
| | | Initial | Only | and Filtration | Centrifugation |
| | Molar | Concentration | Cᵣ (mmol/L) | C_{f} (mmol/L) | C_{c} (mmol/L) |
| Rhamnolipid | Ratio | (mmol/L) | (Rᵣ) | (R_{f}) | (R_{c}) |
| Rha C6 | 2 to 1 | 0.298 ± 0.005 | 0.325 ± 0.026 | 0.143 ± 0.062 | 0.324 ± 0.018 |
| | | | (NR) | (51.8%) | (NR) |
| | 5 to 1 | | 0.282 ± 0.026 | 0.106 ± 0.026 | 0.320 ± 0.024 |
| | | | (5.2%) | (64.4%) | (NR) |
| | 10 to 1 | | 0.332 ± 0.040 | 0.114 ± 0.064 | 0.294 ± 0.010 |
| | | | (NR) | (61.6%) | (1.1%) |
| Rha C10 | 2 to 1 | 0.280 ± 0.009 | 0.282 ± 0.009 | 0.096 ± 0.025 | 0.228 ± 0.013 |
| | | | (NR) | (65.7%) | (18.6%) |
| | 5 to 1 | | 0.246 ± 0.016 | 0.015 ± 0.010 | 0.088 ± 0.003 |
| | | | (12.3%) | (94.6%) | (68.7%) |
| | 10 to 1 | | 0.190 ± 0.004 | 0.022 ± 0.028 | 0.051 ± 0.010 |
| | | | (32.2%) | (92.1%) | (81.7%) |
| Rha C14 | 2 to 1 | 0.298 ± 0.005 | 0.247 ± 0.034 | 0.164 ± 0.058 | 0.221 ± 0.014 |
| | | | (16.9%) | (44.9%) | (25.8%) |
| | 5 to 1 | | 0.215 ± 0.024 | 0.044 ± 0.007 | 0.094 ± 0.002 |
| | | | (27.7%) | (85.3%) | (68.5%) |
| | 10 to 1 | | 0.314 ± 0.016 | 0.0 ± 0.000 | 0.006 ± 0.001 |
| | | | (NR) | 1.0 (99.9%) | (98.0%) |
| Rha C18 | 2 to 1 | 0.298 ± 0.005 | 0.249 ± 0.009 | 0.116 ± 0.029 | 0.228 ± 0.013 |
| | | | (16.5%) | (61.1%) | (23.4%) |
| | 5 to 1 | | 0.121 ± 0.009 | 0.001 ± 0.002 | 0.088 ± 0.003 |
| | | | (59.4%) | (99.5%) | (70.6%) |
| | 10 to 1 | | 0.331 ± 0.024 | 0.004 ± 0.001 | 0.051 ± 0.010 |
| | | | (NR) | (98.8%) | (82.8%) |
| Rha C10C10 | 2 to 1 | 0.268 ± 0.002 | 0.144 ± 0.006 | 0.085 ± 0.005 | 0.155 ± 0.002 |
| | | | (46.4%) | (68.5%) | (42.1%) |
| | 5 to 1 | | 0.219 ± 0.007 | 0.001 ± 0.000 | 0.003 ± 0.000 |
| | | | (18.4%) | (99.7%) | (98.9%) |
| | 10 to 1 | | 0.257 ± 0.004 | 0.030 ± 0.002 | 0.035 ± 0.003 |
| | | | (4.0%) | (88.7%) | (86.9%) |
| Rha C12C12 | 2 to 1 | 0.268 ± 0.002 | 0.184 ± 0.014 | 0.119 ± 0.007 | 0.164 ± 0.013 |
| | | | (31.5%) | (55.7%) | (38.7%) |
| | 5 to 1 | | 0.197 ± 0.006 | 0.044 ± 0.009 | 0.074 ± 0.003 |
| | | | (26.4%) | (83.7%) | (72.4%) |
| | 10 to 1 | | 0.263 ± 0.007 | 0.035 ± 0.001 | 0.057 ± 0.004 |
| | | | (2.0%) | (86.9%) | (78.7%) |
| Rha C14C14 | 2 to 1 | 0.268 ± 0.002 | 0.158 ± 0.016 | 0.105 ± 0.013 | 0.160 ± 0.002 |
| | | | (41.1%) | (60.7%) | (40.3%) |
| | 5 to 1 | | 0.143 ± 0.005 | 0.022 ± 0.004 | 0.060 ± 0.003 |
| | | | (46.7%) | (92.0%) | (77.5%) |
| | 10 to 1 | | 0.246 ± 0.014 | 0.031 ± 0.008 | 0.029 ± 0.003 |
| | | | (8.1%) | (88.3%) | (89.1%) |

TABLE 5
Concentrations (mmol/L) of magnesium in the reaction tube initially and after the
three treatments. The molar ratio (column 2) is rhamnolipid:magnesium. The efficiency
of removal is presented as percent in parentheses below the concentrations.

| | | | | | Treatment 3: |
|---|---|---|---|---|---|
| | | | Treatment 1: | Treatment 2: | Rhamnolipid |
| | | | Rhamnolipid | Rhamnolipid | and |
| | | Initial | Only | and Filtration | Centrifugation |
| | Molar | Concentration | Cᵣ (mmol/L) | C_{f} (mmol/L) | C_{c} (mmol/L) |
| Rhamnolipid | Ratio | (mmol/L) | (Rᵣ) | (R_{f}) | (R_{c}) |
| Rha C6 | 2 to 1 | 0.268 ± 0.008 | 0.247 ± 0.014 | 0.202 ± 0.016 | 0.265 ± 0.010 |
| | | | (7.6%) | (24.5%) | (0.9%) |
| | 5 to 1 | | 0.248 ± 0.013 | 0.178 ± 0.020 | 0.255 ± 0.001 |
| | | | (7.3%) | (33.4%) | (4.9%) |
| | 10 to 1 | | 0.258 ± 0.011 | 0.165 ± 0.001 | 0.272 ± 0.016 |
| | | | (3.6%) | (38.2%) | (NR) |
| Rha C10 | 2 to 1 | 0.501 ± 0.009 | 0.486 ± 0.021 | 0.328 ± 0.036 | 0.497 ± 0.014 |
| | | | (3.1%) | (34.6%) | (0.8%) |
| | 5 to 1 | | 0.492 ± 0.007 | 0.283 ± 0.059 | 0.500 ± 0.008 |
| | | | (1.8%) | (43.6%) | (0.2%) |
| | 10 to 1 | | 0.503 ± 0.004 | 0.317 ± 0.073 | 0.490 ± 0.004 |
| | | | (NR) | (36.7%) | (2.2%) |
| Rha C14 | 2 to 1 | 0.268 ± 0.008 | 0.291 ± 0.005 | 0.147 ± 0.045 | 0.292 ± 0.008 |
| | | | (NR) | (45.0%) | (NR) |
| | 5 to 1 | | 0.300 ± 0.018 | 0.088 ± 0.042 | 0.279 ± 0.016 |
| | | | (NR) | (66.9%) | (NR) |
| | 10 to 1 | | 0.302 ± 0.017 | 0.009 ± 0.004 | 0.219 ± 0.021 |
| | | | (NR) | (96.6%) | (NR) |
| Rha C18 | 2 to 1 | 0.268 ± 0.008 | 0.265 ± 0.002 | 0.169 ± 0.015 | 0.216 ± 0.002 |
| | | | (12.1%) | (37.0%) | (20.5%) |
| | 5 to 1 | | 0.215 ± 0.002 | 0.056 ± 0.012 | 0.141 ± 0.001 |
| | | | (19.5%) | (79.0%) | (47.4%) |
| | 10 to 1 | | 0.304 ± 0.001 | 0.017 ± 0.002 | 0.107 ± 0.004 |
| | | | (NR) | (93.7%) | (60.1%) |
| Rha C10C10 | 2 to 1 | 0.268 ± 0.008 | 0.297 ± 0.001 | 0.127 ± 0.007 | 0.292 ± 0.002 |
| | | | (NR) | (52.4%) | (NR) |
| | 5 to 1 | | 0.282 ± 0.023 | 0.023 ± 0.003 | 0.300 ± 0.002 |
| | | | (NR) | (91.4%) | (NR) |
| | 10 to 1 | | 0.308 ± 0.004 | 0.008 ± 0.002 | 0282 ± 0.004 |
| | | | (NR) | (97.1%) | (NR) |
| Rha C12C12 | 2 to 1 | 0.268 ± 0.008 | 0.281 ± 0.002 | 0.182 ± 0.055 | 0.276 ± 0.005 |
| | | | (NR) | (32.1%) | (NR) |
| | 5 to 1 | | 0.285 ± 0.005 | 0.135 ± 0.074 | 0.273 ± 0.014 |
| | | | (NR) | (49.4%) | (NR) |
| | 10 to 1 | | 0.285 ± 0.012 | 0.008 ± 0.004 | 0.266 ± 0.012 |
| | | | (NR) | (97.1%) | (0.4%) |
| Rha C14C14 | 2 to 1 | 0.268 ± 0.008 | 0.279 ± 0.002 | 0.152 ± 0.077 | 0.270 ± 0.004 |
| | | | (NR) | (43.2%) | (NR) |
| | 5 to 1 | | 0.270 ± 0.006 | 0.170 ± 0.098 | 0.269 ± 0.013 |
| | | | (NR) | (36.4%) | (NR) |
| | 10 to 1 | | 0.275 ± 0.009 | 0.009 ± 0.006 | 0.264 ± 0.010 |
| | | | (NR) | (96.8%) | (1.5%) |

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

### Example 2.2: Glycolipid Facilitated Chemical Precipitation of Metals and Rees

Surfactants: Each of the solutions were made by dissolving a single glycolipid (GlycoSurf Inc., Salt Lake City, >95% purity) in nanopure water (≥18 MΩ-cm) then adjusting the pH to 6.90±0.05 with HNO₃ and NaOH. Nine different structural variants were selected to examine the effect of the headgroup and number and length of the hydrophobic tail(s) on glycolipids as a chemical precipitant (Table 6). These solutions were made in advance and stored at 4° C. for up to one month and brought to room temperature (˜25° C.) before each experiment.

TABLE 6
The glycolipids structures tested.

| | Abbrevia- | Molecular | Chemical | |
|---|---|---|---|---|
| Glycolipid | tion | Weight | Formula | Structure |
| Galactolipid C10 | Gal-C10 | 350.41 | C₁₆H₃₀O₈ | 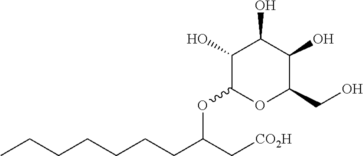 [SMILES: CCCCCCCC(CC(=O)O)OC1O[C@H](CO)[C@H](O)[C@H](O)[C@H]1O] |
| Galactolipid C14 | Gal-C14 | 406.52 | C₂₀H₃₈O₈ | 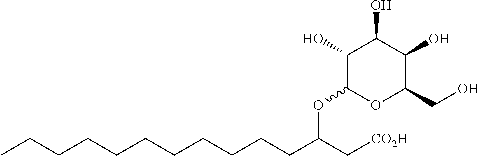 [SMILES: CCCCCCCCCCCC(CC(=O)O)OC1O[C@H](CO)[C@H](O)[C@H](O)[C@H]1O] |
| Galactolipid C18 | Gal-C18 | 514.60 | C₂₄H₄₂O₈ | 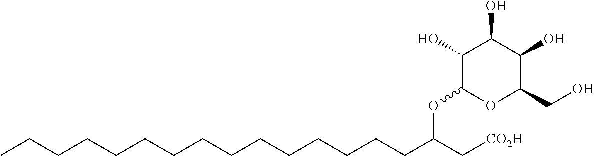 [SMILES: CCCCCCCCCCCCCCCC(CC(=O)O)OC1O[C@H](CO)[C@H](O)[C@H](O)[C@H]1O] |
| Galactolipid C10-C10 | Gal-C10- C10 | 520.66 | C₂₆H₄₈O₁₀ | 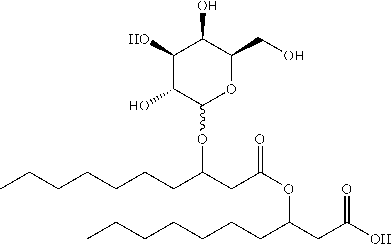 [SMILES: CCCCCCCC(CC(=O)O)OC(=O)CC(CCCCCCC)OC1O[C@H](CO)[C@H](O)[C@H](O)[C@H]1O] |
| Xylolipid C10 | Xyl-C10 | 320.38 | C₁₅H₂₈O₇ | 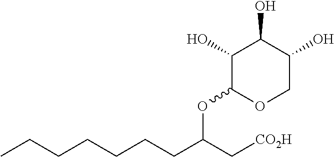 [SMILES: CCCCCCCC(CC(=O)O)OC1OC[C@@H](O)[C@H](O)[C@H]1O] |
| Xylolipid C14 | Xyl-C14 | 376.49 | C₁₉H₃₆O₇ | 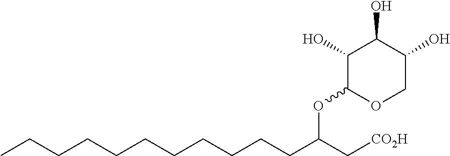 [SMILES: CCCCCCCCCCCC(CC(=O)O)OC1OC[C@@H](O)[C@H](O)[C@H]1O] |
| Xylolipid C18 | Xyl-C18 | 430.57 | C₂₃H₄₂O₇ | 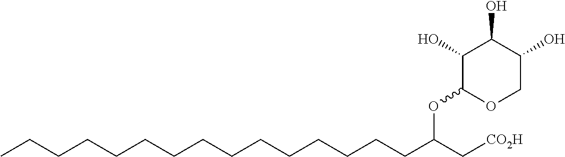 [SMILES: CCCCCCCCCCCCCCCC(CC(=O)O)OC1OC[C@@H](O)[C@H](O)[C@H]1O] |
| Xylolipid C10-C10 | Xyl-C10- C10 | 490.93 | C₂₅H₄₂O₉ | 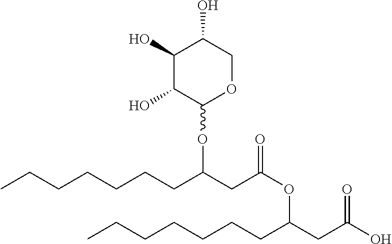 [SMILES: CCCCCCCC(CC(=O)O)OC(=O)CC(CCCCCCC)OC1OC[C@@H](O)[C@H](O)[C@H]1O] |
| Xylolipid C1414 | Xyl-C14- C14 | 602.85 | C₃₃H₆₂O₉ | 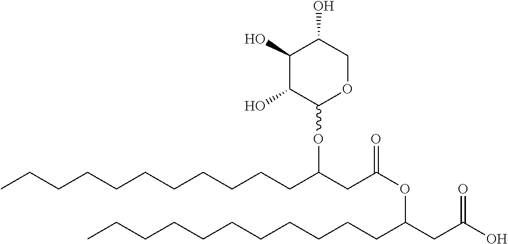 [SMILES: CCCCCCCCCCCC(CC(=O)O)OC(=O)CC(CCCCCCCCCCC)OC1OC[C@@H](O)[C@H](O)[C@H]1O] |

Metals: Three metals were tested for binding with the glycolipids: lead as Pb(NO₃)₃ (Fisher Scientific, Waltham, MA, 99% purity), lanthanum as La(NO₃)₃ 6(H₂O) (Sigma-Aldrich, St. Louis, MO, 99.99% purity), and magnesium as Mg(NO₃)₂ 6(H₂O) (Sigma-Aldrich, St. Louis, MO, 99% purity. Metal solutions (5 mM) were made using nanopure water (≥18 MΩ-cm) and were not pH balanced.

### Methods:

Each metal (Pb²⁺, La³⁺, and Mg²⁺) was evaluated separately with each of the nine glycolipids. For every experiment, a metal solution was mixed with a glycolipid solution in a 1.7 ml polypropylene centrifuge tube at varying molar ratios (glycolipid:metal): 0:1 (glycolipid-free control), 2:1, 5:1, and 10:1. The reactions were mixed in the order of nanopure water (≥18 MΩ-cm), glycolipid solution, then metal solution. The metal and glycolipid solutions were continuously stirred as they were aliquoted into the reaction tubes. All experiments were performed in triplicate.

The reaction tubes were rotated overnight (16 to 24 h) and then three samples were collected from each tube to measure metal removal from solution: (1) no further treatment; (2) an additional filtration step; and (3) an additional centrifugation step. Briefly, for the glycolipid adsorption treatment samples all tubes were vortexed for approximately 5 sec immediately before sampling to ensure that non-adsorbed glycolipid-metal complexes were mixed into the solution column. After mixing, 0.1 ml of the reaction solution was aliquoted into a dilution tube of 2% trace metal grade nitric acid for analysis. This sample measures the loss of metal due to the formation of hydrophobic scums or adsorbed complexes not attributable to the subsequent filtration or centrifugation treatments.

Samples for the filtration treatment were vortexed for 5 sec, then a 0.6 ml aliquot was collected with a 1 ml syringe. A GHP acrodisc disk filter (hydrophilic polypropylene membrane, 0.45 pm pore size, 25 mm disc diameter) from Pall Life Sciences (Port Washington, New York) or PTFE disk filters (hydrophobic polytetrafluoroethylene membrane, 0.45 μm pore size, 25 mm disc diameter) from Fisher Scientific (Waltham, MA) was then placed onto the end of the syringe and approximately 0.1 ml of filtrate was collected into a dilution tube for analysis. The centrifuge sample was collected by centrifuging the remaining reaction solution at 10,000 RCF for 15 min. Approximately 0.1 ml of the supernatant was aliquoted into a dilution tube for analysis. Following collection, the metal content in all samples was analyzed at the Arizona Laboratory for Emerging Contaminants (ALEC) at the University of Arizona using inductively coupled plasma mass spectrometry.

Calculations: The efficiency of metal removal (R) was determined for each of the three treatments: Rₐ for the active mixing/adsorption treatment, R_{f} for the glycolipid and filtration treatment, and R_{c} for the glycolipid and centrifugation treatment. R was determined by comparing the solution metal concentration of the treatment sample (Cₙₜ for the glycolipid with no further treatment sample, C_{f} for the glycolipid and filtration sample, and C_{c} for the glycolipid and centrifugation sample) to the solution metal concentration of a glycolipid-free control (Cᵢ) that underwent the same handling and processing steps. The equations for these calculations are shown below:

$${R}_{a} = 100 ( 1 - \frac{{C}_{nt}}{{C}_{i}} ) \qquad Eq . 2$$

$${R}_{f} = 100 ( 1 - \frac{{C}_{f}}{{C}_{i}} ) \qquad Eq . 3$$

$${R}_{c} = 100 ( 1 - \frac{{C}_{c}}{{C}_{i}} ) \qquad Eq . 4$$

### Results

Metal Recovery Following Precipitation without Further Treatment: The sample that received no further treatment following the addition of the glycolipid provided insight into the ability of the glycolipids to precipitate Pb²⁺, La³⁺ and Mg²⁺ from single metal solutions. This was determined both visually and with measurement of the metal concentration in the aqueous phase. There were three types of visual observations made which varied with the glycolipid treatment (i.e., the glycolipid structure and concentration). The first was the formation of glycolipid-metal complexes as hydrophobic scums along the reaction tube during active mixing. The second was cloudy solutions of dispersed precipitates and the third was clear solutions without any visible precipitation. For example, these behaviors were all observed in the La³⁺-xylolipid reactions. Specifically, Xyl-C14 did not result in any visible precipitation and therefore had a clear solution with all molar ratios. On the other hand, Xyl-C18 had precipitation build-up on the walls of the reaction tubes at the 2:1 ratio while cloudy solutions were observed at the 5:1 and 10:1 ratios. The Xyl-C14-C14 treatment had different precipitation results; a clear solution was observed at the 2:1 ratio while the 5:1 and 10:1 ratios resulted in precipitation on the reaction tube surfaces.

The effectiveness of metal removal for the four galactolipids (Gal-C10, Gal-C14, Gal-C18, and Gal-C10-C10) was observed in the order of La³⁺>Pb²⁺»Mg²⁺ with maximum removals reaching 57.9%, 45.1% and 11.6%, respectively (Tables 3.2-3.4). Maximum removal was achieved with Gal-C18 at the 2:1 ratio for Pb²⁺, and Gal-C14 at the 5:1 ratio for both La³⁺ and Mg²⁺. Pb²⁺ was removed in the order of Gal-C18>Gal-C10>Gal-C10-C10>Gal-C14 with maximum removals at 45%, 38%, 24% and 1.5% respectively. For La³⁺, three galactolipids (Gal-C10, Gal-C14 and Gal-C10-C10) removed between 53% and 58% while Gal-C18 did not remove any La³⁺ with this treatment method. Removal for Mg²⁺ was very low with the galactolipids tested, all efficiencies were under 12%.

For xylolipids (Xyl-C10, Xyl-C14, Xyl-C18, Xyl-C10-C10, and Xyl-C14-C14) removal was in the order of La³⁺»Pb²⁺>Mg²⁺ with maximum removals of 77.4%, 30.6%, and 17.1%, respectively (Tables 3.2-3.4). For Pb²⁺, Xyl-C10, Xyl-C10-C10, and Xyl-C14-C14 achieved the highest amounts of removal, approximately 30% while Xyl-C18 and Xyl-C14 removed under 14% and 7%, respectively. For La³⁺, Xyl-C18, Xyl-C10-C10 and Xyl-C14 achieved better removals with 77.4%, 51.6%, and 42.6%, respectively. The other xylolipids (Xyl-C14-C14 and Xyl-C10) each removed less than 15%. Xyl-C14-C14, which removed 17.1% of Mg²⁺ from solution, was the only xylolipid to remove more than 7% of metal from solution.

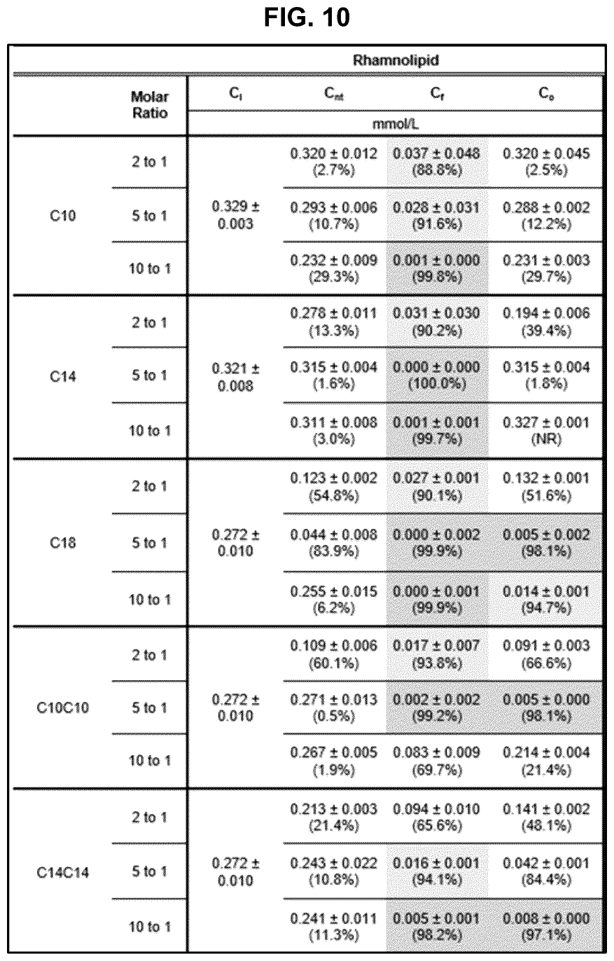

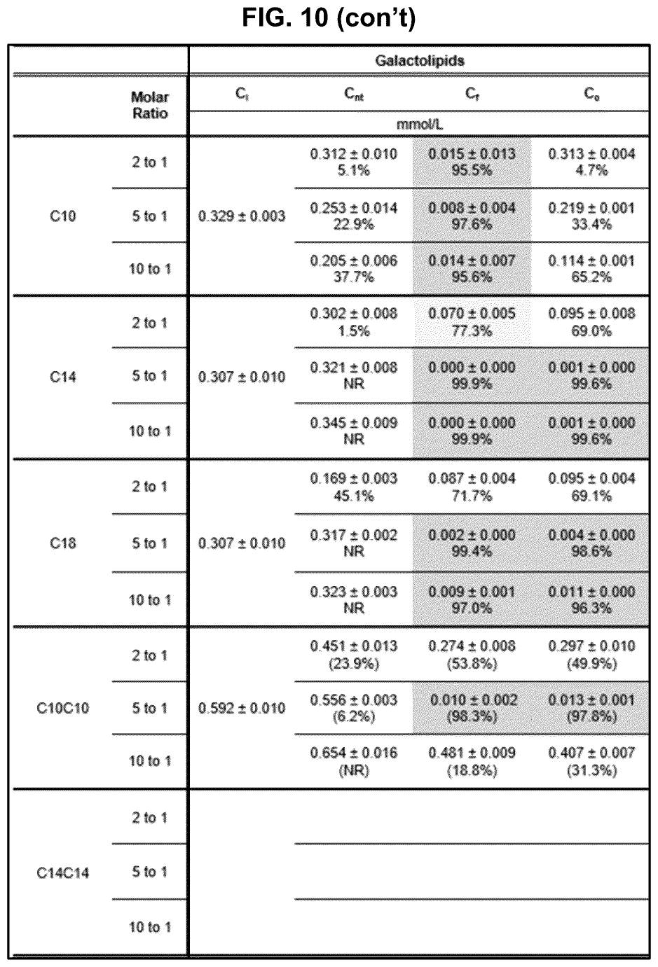

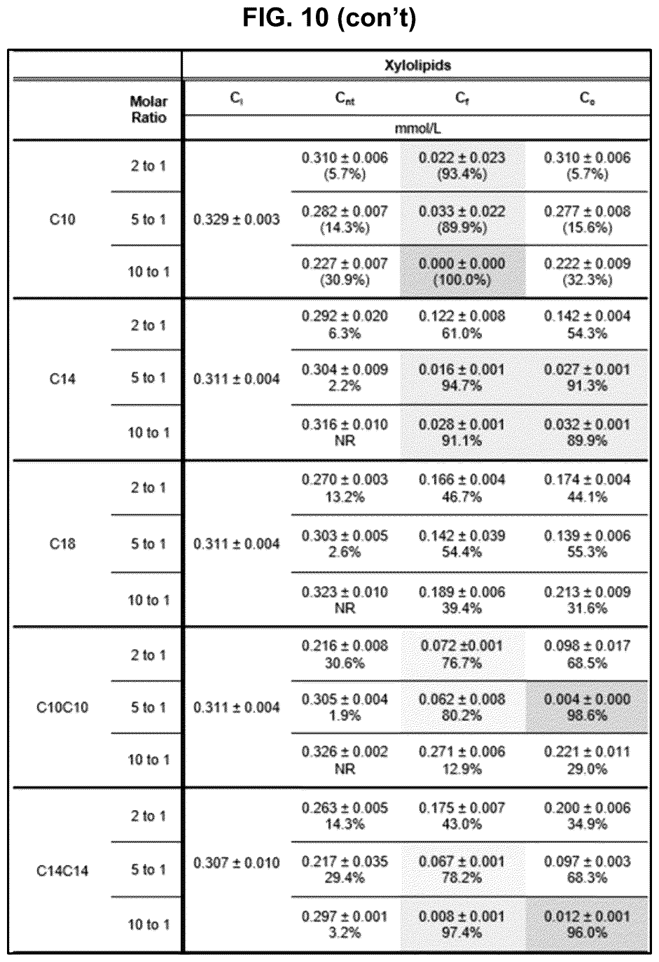

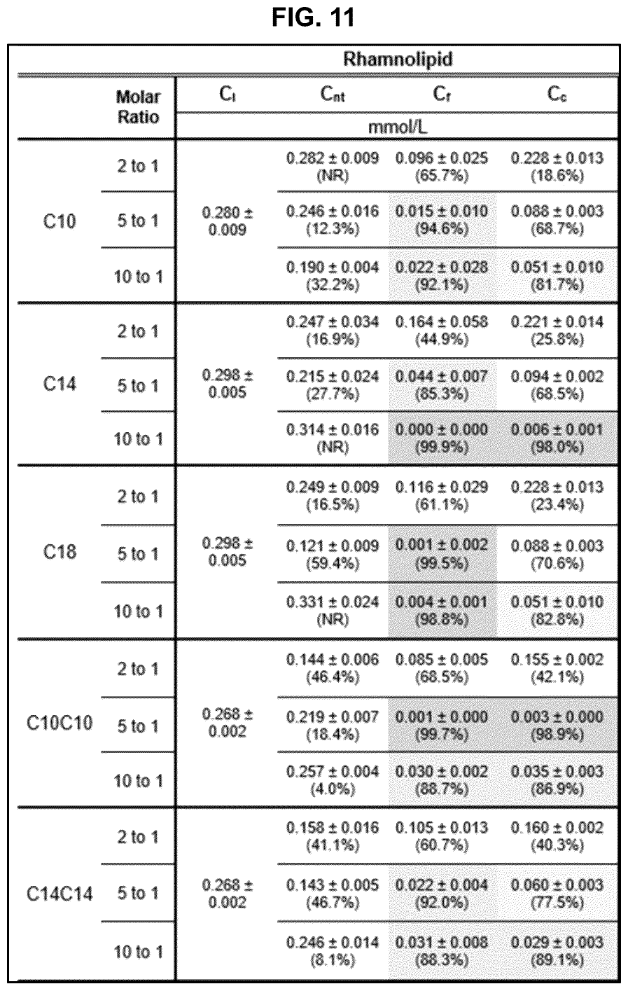

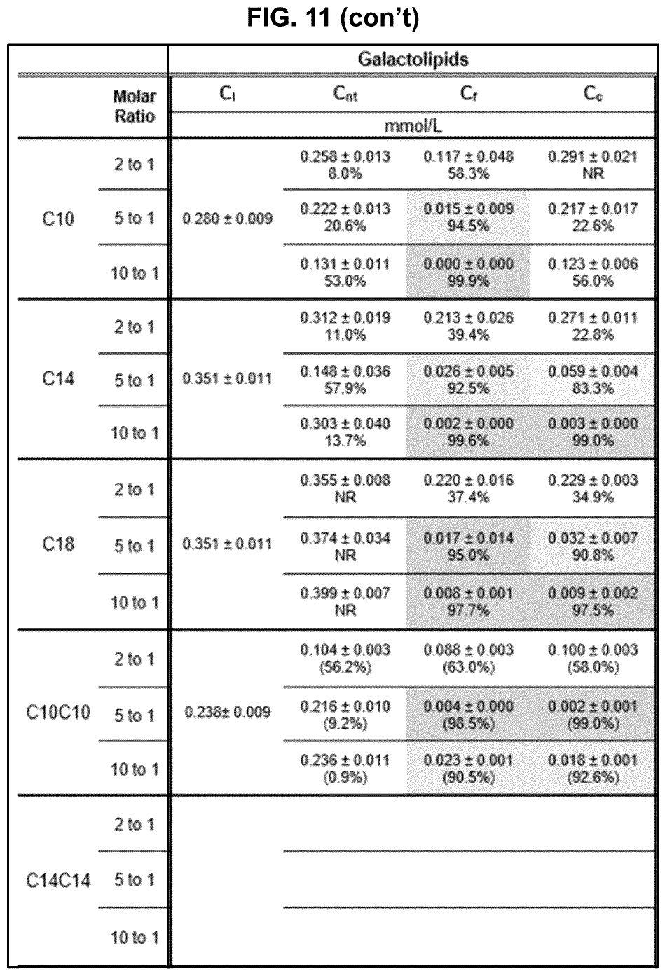

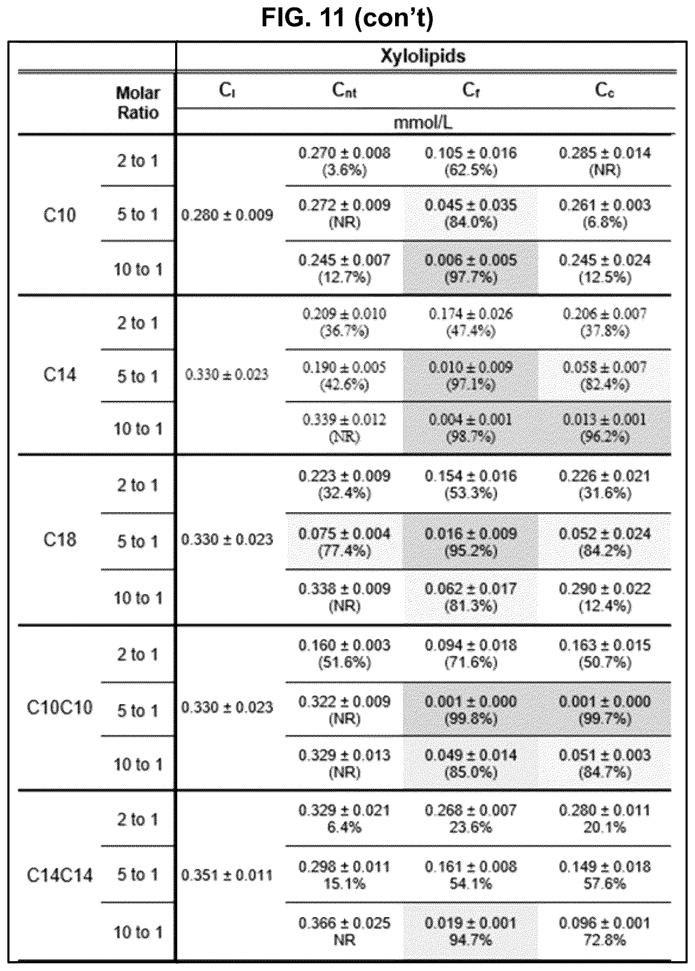

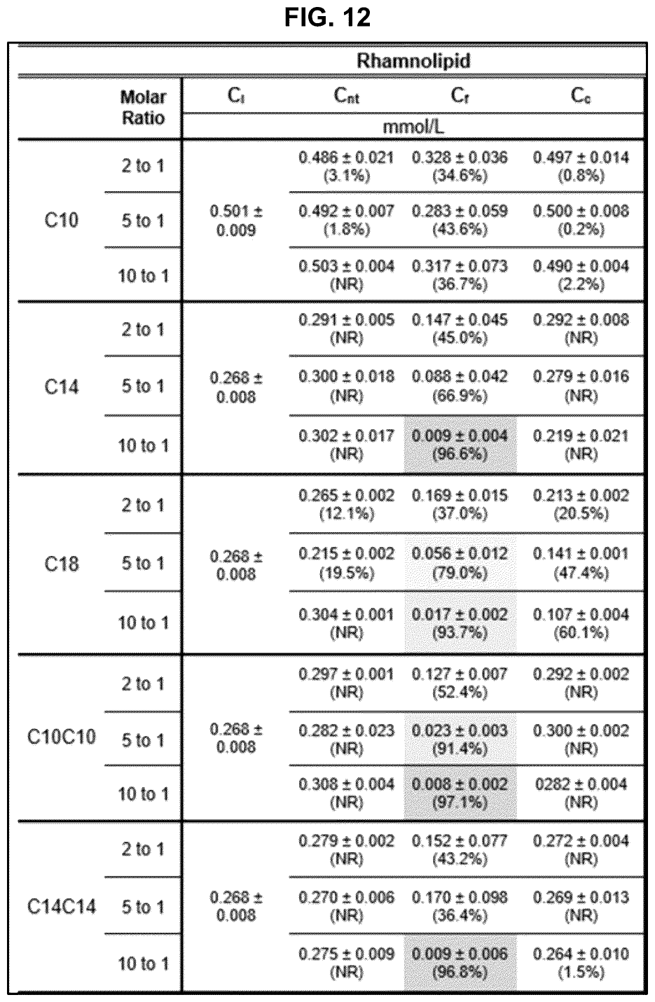

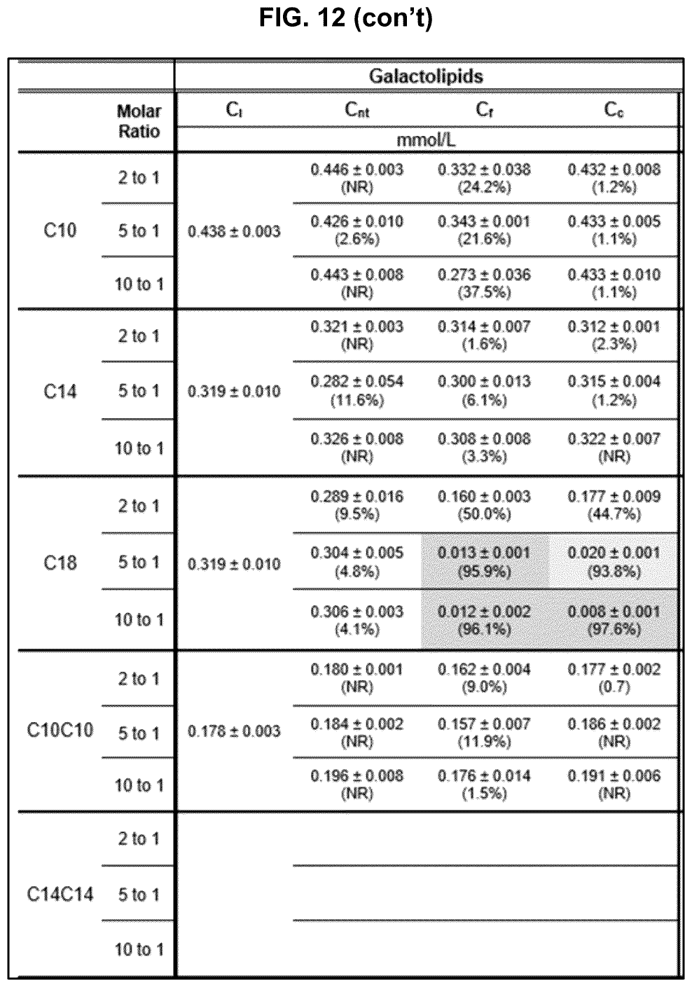

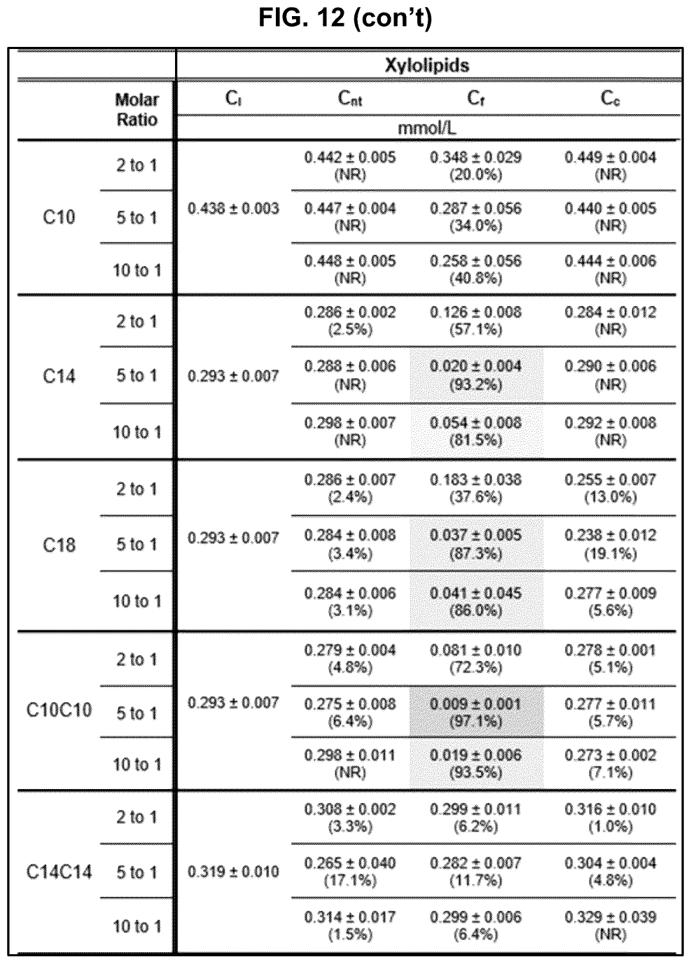

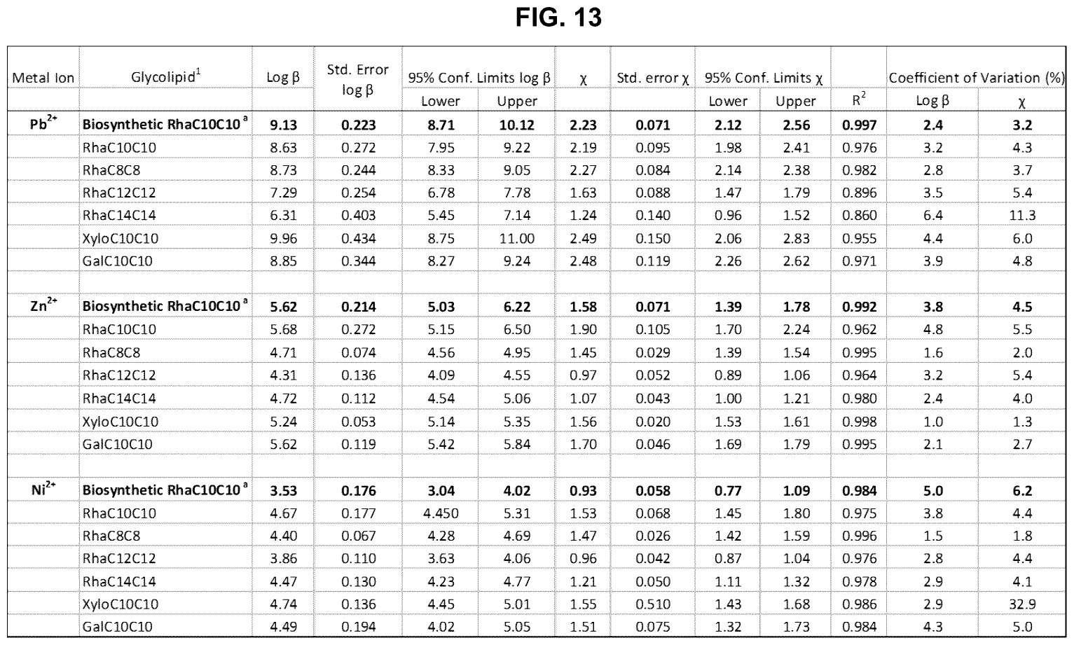

Metal Recovery: Precipitation Followed by Filtration: An additional filtration step was added to enhance the removal of the glycolipid-metal complexes from solution. Filtration increased the removal efficiencies for all metals tested in the order of Pb²⁺>La³⁺>Mg²⁺. Specifically, the four galactolipids removed from 18.8% to 99.9% of Pb²⁺, depending on the galactolipid and the molar ratio tested (FIG. 10). Each galactolipid tested removed >97% of Pb²⁺ at the 5:1 molar ratio. Performance at the 10:1 ratio was similarly high among the galactolipids tested except for the Gal-C10-C10 which only removed 18.8%. At the 2:1 molar ratio, with the exception of the Gal-C10 (95.5% removal), the galactolipids did not perform as well (53.8-77.3% removal). For La³⁺, removal ranged between 37.4% to 99.9% with all of the 5:1 ratios removing over 92.5% (FIG. 11). For the 10:1 ratio, removal was over 97.7% with the exception of the Gal-C10-C10 which removed 90.5%. Finally, for Mg²⁺, removal was much lower, ranging from 1.5% to 50.0%, with two exceptions (FIG. 12) The exceptions were the Gal-C18 which removed 95.9% and 96.1% of Mg²⁺ at the 5:1 and 10:1 molar ratios, respectively.

Similar to the galactolipids, the xylolipids performed better with the addition of a filtration step. For Pb²⁺, three of the xylolipids achieved >94.7% Pb²⁺ removal, with the Xyl-C10 and the Xyl-C14-C14 performing best at the 10:1 molar ratio and the Xyl-C14 performing best at the 5:1 molar ratio (FIG. 10). The remaining xylolipids, Xyl-C18 was the least effective at removing Pb²⁺ from solution (<54.4%). In contrast, for La³⁺, all five xylolipids tested showed >94.7% removal at either the 5:1 or 10:1 ratios (FIG. 11). Performance was not as good at the 2:1 ratios, all removed less than 70%. Finally, for Mg²⁺, only two of the xylolipids showed removals of >90%, the Xyl-C1010 (at the 5:1 and 10:1 molar ratios) and the Xyl-C14 (at the 5:1 molar ratio) (FIG. 12).

Metal Recovery: Precipitation Followed by Centrifugation: Centrifugation was examined as an alternative added step to aid in the removal of the glycolipid complex from solution. The addition of the centrifugation step greatly increased the removal of metals from solution. For the galactolipids tested, Pb²⁺ removal ranged from 4.7 to 99.6% (FIG. 10). Three galactolipids removed over 96% of Pb²⁺ from solution. Gal-C14 and Gal-C18 achieved this removal at both the 5:1 and 10:1 molar ratios while the third galactolipid, Gal-C10-C10, only did so at the 10:1 molar ratio. For La³⁺, removal ranged from non-detectable to 99.0%. The pattern of removal of La³⁺ by the galactolipids was similar to Pb²⁺ except that the Gal-C10-C10 performed well at both the 5:1 and 10:1 molar ratios. The least effective galactolipid for both Pb²⁺ and La³⁺ was the Gal-C10. In general removal of Mg²⁺ was lower than for Pb²⁺ and La³⁺. However, the Gal-C18 removed 93.8% and 97.6% Mg²⁺ for the 5:1 and 10:1 molar ratios respectively. The Gal-C10, Gal-C14, and Gal-C10-C10 all removed less than 3% Mg²⁺. Across the three metals, the most effective molar ratio was generally in the order of 10:1>5:1»2:1.

Among the five xylolipids tested, three of the molecules (Xyl-C14, Xyl-C10-C10, and Xyl-C14-C14) were able to achieve >90% removal of Pb²⁺. For La³⁺, two of the xylolipids had >90% removal, the Xyl-C14 and the Xyl-C10-C10. None of the xylolipids tested removed Mg²⁺ beyond 19.1%. It is noted that the Xyl-C10 did not remove any metal more than 30% at any molar ratio using centrifugation.

### EMBODIMENTS

The following embodiments are intended to be illustrative only and not to be limiting in any way.

Embodiment 1: A glycolipid-heavy metal ion complex, comprising: at least one glycolipid of the formula: A-B, wherein A is selected from the group consisting of a monosaccharide, a disaccharide, and a derivative thereof; and B is a moiety of the formula:

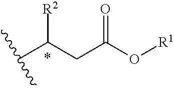

wherein * is a chiral center; R² is C₁-C₂₀ hydrocarbon optionally having 1, 2, or 3 carbon-carbon double bonds; and R¹ is H, C₁-C₂₀ hydrocarbon optionally having 1, 2, or 3 carbon-carbon double bonds, or a moiety of the formula:

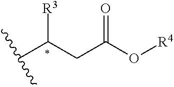

wherein R³ is C₁-C₂₀ hydrocarbon optionally having 1, 2, or 3 carbon-carbon double bonds, and R⁴ is H or C₁-C₂₀ hydrocarbon optionally having 1, 2, or 3 carbon-carbon double bonds; and a heavy metal ion, wherein said heavy metal ion comprises an ion of uranium, Y, Ce, Pr, Nd, Pm, Sm, Eu, Gd, Tb, Dy, Ho, Er, Tm, Yb, Lu, La, Cu, Ag, Au, Pd, Pt, Pb, Cd, Zn, Tl, Hg, or a combination thereof; wherein the at least one glycolipid and the heavy metal ion together form the glycolipid-heavy metal ion complex.

Embodiment 2: The glycolipid-heavy metal ion complex of embodiment 1, wherein the heavy metal ion comprises an ion of uranium, Y, La, Ce, Pr, Nd, Pm, Sm, Eu, Gd, Tb, Dy, Ho, Er, Tm, Yb, Lu, lead (Pb), or a combination thereof.

Embodiment 3: The glycolipid-heavy metal ion complex of embodiment 1, wherein the at least one glycolipid and heavy metal ion are present in the complex in a ratio ranging from about 1:1 to 4:1, 1:1 to 3:1, or 1:1 to 2:1.

Embodiment 4: The glycolipid-heavy metal ion complex of embodiment 1, wherein A is a monosaccharide or a thiol derivative thereof.

Embodiment 5: The glycolipid-heavy metal ion complex of embodiment 4, wherein B is attached to the hydroxyl group of the anomeric carbon or a thiol derivative thereof of said monosaccharide.

Embodiment 6: The glycolipid-heavy metal ion complex of embodiment 1, wherein said monosaccharide is selected from the group consisting of glucose, fructose, galactose, rhamnose, arabinose, xylose, fucose, and a thiol derivative thereof.

Embodiment 7: The glycolipid-heavy metal ion complex of embodiment 1, wherein A is a disaccharide or a thiol derivative thereof.

Embodiment 8: The glycolipid-heavy metal ion complex of embodiment 7, wherein said disaccharide comprises 1,4-linkage or 1,6-linkage between two monosaccharides.

Embodiment 9: The glycolipid-heavy metal ion complex of embodiment 7, wherein said disaccharide is selected from the group consisting of lactose, maltose, melibiose, cellobiose, rutinose, and a thiol derivative thereof.

Embodiment 10: The glycolipid-heavy metal ion complex of embodiment 1, wherein R² is CH₃—(CH₂)ₙ— and R³ is CH₃—(CH₂)ₘ—, and wherein each of m and n is independently an integer from 4 to 12.

Embodiment 11: The glycolipid-heavy metal ion complex of embodiment 1, wherein B is of the formula:

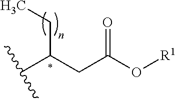

wherein *, and R¹ are those defined in embodiment 1, and wherein n is an integer in a range from 0 to 19.

Embodiment 12: The glycolipid-heavy metal ion complex of embodiment 11, wherein R¹ is a moiety of the formula:

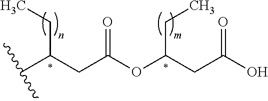

wherein m and n are independently an integer in a range from 0 to 19.

Embodiment 13: The glycolipid-heavy metal ion complex of embodiment 12, wherein each of m and n is independently 6, 8, or 10.

Embodiment 14: The glycolipid-heavy metal ion complex of embodiment 1, wherein R¹ is C₆₋₂₀ alkyl.

Embodiment 15: The glycolipid-heavy metal ion complex of any one of embodiments 1 to 13, further comprising at least one C1 to C6 alcohol frother additive to stabilize foam structure, thereby facilitating the separation of the glycolipid-heavy metal ion complex.

Embodiment 16: The glycolipid-heavy metal ion complex of any one of embodiments 1 to 15, wherein the glycolipid-heavy metal ion complex is substantially insoluble at 25° C. at a pH in a range from about 3 to about 12

Embodiment 17: A composition comprising: at least one glycolipid of the formula: A-B, wherein A is selected from the group consisting of a monosaccharide, a disaccharide, and a derivative thereof; and B is a moiety of the formula:

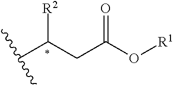

wherein * is a chiral center; R² is C₁-C₂₀ hydrocarbon optionally having 1, 2, or 3 carbon-carbon double bonds; and R¹ is H, C₁-C₂₀ hydrocarbon optionally having 1, 2, or 3 carbon-carbon double bonds, or a moiety of the formula:

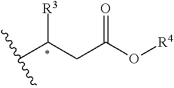

wherein R³ is C₁-C₂₀ hydrocarbon optionally having 1, 2, or 3 carbon-carbon double bonds, and R⁴ is H or C₁-C₂₀ hydrocarbon optionally having 1, 2, or 3 carbon-carbon double bonds; a frothing agent, wherein the frothing agent comprises a C1-C3 alcohol; and water.

Embodiment 18: The composition of embodiment 17, wherein a concentration of the glycolipid in the water is in a range of from about 50 μM to about 10 mM.

Embodiment 19: The composition of embodiment 17, wherein a concentration of the frothing agent in the water is in a range of from about 0% to about 2% (v/v).

Embodiment 20: The composition of embodiment 17, wherein a ratio of frothing agent to glycolipid is in a range from about 0:1 to about 1500:1.

Embodiment 21: The composition of any one of embodiments 17 to 20, wherein the frothing agent is ethanol.

Embodiment 22: A method for removing a heavy metal ion from a sample, said method comprising: contacting said sample with an aqueous solution comprising a glycolipid to form a glycolipid-heavy metal ion complex; and separating said glycolipid-heavy metal ion complex, thereby removing said heavy metal ion from said sample.

Embodiment 23: The method of embodiment 22, wherein said sample comprises any of soil, groundwater, industrial wastewater, acid mine drainage, mining (both coal and hard rock) process waters and solid residuals, produced waters, electroplating solutions, coal combustion process waters, landfill leachates, e-waste, and fly ash.

Embodiment 24: The method of embodiment 22, wherein said heavy metal ion comprises an ion of uranium, Y, Ce, Pr, Nd, Pm, Sm, Eu, Gd, Tb, Dy, Ho, Er, Tm, Yb, Lu, La, Cu, Ag, Au, Pd, Pt, Pb, Cd, Zn, Tl, Hg, or a combination thereof.

Embodiment 25: The method of embodiment 22, wherein said glycolipid is of the formula: A-B, wherein A is selected from the group consisting of a monosaccharide, a disaccharide, and a derivative thereof; and B is a moiety of the formula:

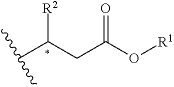

wherein * is a chiral center; R² is C₁-C₂₀ hydrocarbon optionally having 1, 2, or 3 carbon-carbon double bonds; and R¹ is H, C₁-C₂₀ hydrocarbon optionally having 1, 2, or 3 carbon-carbon double bonds, or a moiety of the formula:

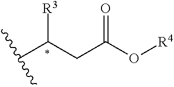

wherein R³ is C₁-C₂₀ hydrocarbon optionally having 1, 2, or 3 carbon-carbon double bonds, and R⁴ is H or C₁-C₂₀ hydrocarbon optionally having 1, 2, or 3 carbon-carbon double bonds.

Embodiment 26: The method of embodiment 25, wherein A is a monosaccharide or a thiol derivative thereof.

Embodiment 27: The method of embodiment 26, wherein B is attached to the hydroxyl group of the anomeric carbon or a thiol derivative thereof of said monosaccharide.

Embodiment 28: The method of embodiment 25, wherein said monosaccharide is selected from the group consisting of glucose, fructose, galactose, rhamnose, arabinose, xylose, fucose, and a thiol derivative thereof.

Embodiment 29: The method of embodiment 25, wherein A is a disaccharide or a thiol derivative thereof.

Embodiment 30: The method of embodiment 29, wherein said disaccharide comprises 1,4-linkage or 1,6-linkage between two monosaccharides.

Embodiment 31: The method of embodiment 29, wherein said disaccharide is selected from the group consisting of lactose, maltose, melibiose, cellobiose, rutinose, and a thiol derivative thereof.

Embodiment 32: The method of embodiment 25, wherein each of m and n is independently an integer from 4 to 12.

Embodiment 33: The method of embodiment 25, wherein B is of the formula:

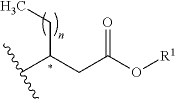

wherein * and R¹ are those defined in embodiment 25 and wherein n is an integer in a range from 0 to 19.

Embodiment 34: The method of embodiment 33, wherein R¹ is a moiety of the formula:

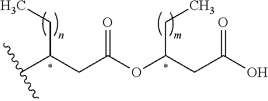

wherein m and n are each independently an integer in a range from 0 to 19.

Embodiment 35: The method of embodiment 34, wherein each of m and n is independently 6, 8, or 10.

Embodiment 36: The method of embodiment 25, wherein R¹ is C₆₋₂₀ alkyl.

Embodiment 37: The method of embodiment 22, wherein said step of separating said glycolipid-heavy metal ion complex comprises any of ion flotation and/or precipitate flotation, precipitation, centrifugation, filtration, settling, or combinations thereof.

Embodiment 38: The method of embodiment 22, wherein said step of separating said glycolipid-heavy metal ion complex comprises ion flotation and/or precipitate flotation of said glycolipid-heavy metal ion complex.

Embodiment 39: The method of embodiment 22, wherein said step of separating said glycolipid-heavy metal ion complex comprises precipitation of said glycolipid-heavy metal ion complex.

Embodiment 40: The method of embodiment 22, wherein said step of separating said glycolipid-heavy metal ion complex comprises centrifugation.

Embodiment 41: The method of embodiment 22, wherein said step of separating said glycolipid-heavy metal ion complex comprises filtration.

Embodiment 42: The method of embodiment 22, wherein said step of separating said glycolipid-heavy metal ion complex comprises gravitational settling.

Embodiment 43: A glycolipid-heavy metal ion complex prepared according to the method of any of embodiments 22-42.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A glycolipid-heavy metal ion complex, comprising:
at least one glycolipid of the formula:
A-B
wherein
A is selected from the group consisting of a monosaccharide, a disaccharide, and a derivative thereof; and
B is a moiety of the formula: 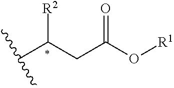
wherein
* is a chiral center;
R² is C₁-C₂₀ hydrocarbon optionally having 1, 2, or 3 carbon-carbon double bonds; and
R¹ is H, C₁-C₂₀ hydrocarbon optionally having 1, 2, or 3 carbon-carbon double bonds, or a moiety of the formula: 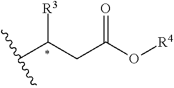
wherein
R³ is C₁-C₂₀ hydrocarbon optionally having 1, 2, or 3 carbon-carbon double bonds, and
R⁴ is H or C₁-C₂₀ hydrocarbon optionally having 1, 2, or 3 carbon-carbon double bonds; and a heavy metal ion,
wherein the at least one glycolipid and the heavy metal ion together form the glycolipid-heavy metal ion complex.

2. The glycolipid-heavy metal ion complex of claim 1, wherein the heavy metal ion comprises an ion of uranium, Y, Ce, Pr, Nd, Pm, Sm, Eu, Gd, Tb, Dy, Ho, Er, Tm, Yb, Lu, La, Cu, Ag, Au, Pd, Pt, Pb, Cd, Zn, Tl, Hg, or a combination thereof.

3. The glycolipid-heavy metal ion complex of claim 1, wherein the at least one glycolipid and heavy metal ion are present in the complex in a ratio ranging from about 1:1 to about 4:1.

4. The glycolipid-heavy metal ion complex of claim 1, wherein A is a monosaccharide or a thiol derivative thereof; wherein said monosaccharide is selected from the group consisting of glucose, fructose, galactose, rhamnose, arabinose, xylose, fucose and a thiol derivative thereof.

5. The glycolipid-heavy metal ion complex of claim 4, wherein B is attached to the hydroxyl group of the anomeric carbon or a thiol derivative thereof of said monosaccharide.

6. The glycolipid-heavy metal ion complex of claim 1, wherein A is a disaccharide or a thiol derivative thereof; wherein said disaccharide is selected from the group consisting of lactose, maltose, melibiose, cellobiose, rutinose, and a thiol derivative thereof.

7. The glycolipid-heavy metal ion complex of claim 6, wherein said disaccharide comprises a 1,4-linkage, or a 1,6-linkage, between two monosaccharides.

8. The glycolipid-heavy metal ion complex of claim 1, wherein R² is CH₃—(CH₂)ₙ— and R³ is CH₃—(CH₂)ₘ—, and wherein each of m and n is independently an integer from 4 to 12.

9. The glycolipid-heavy metal ion complex of claim 1, wherein B is of the formula: 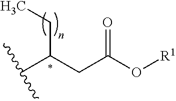
wherein *, and R¹ are those defined in claim 1, and wherein n is an integer in a range from 0 to 19.

10. The glycolipid-heavy metal ion complex of claim 9, wherein R¹ is a moiety of the formula: 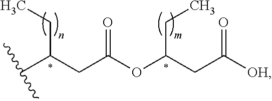
wherein m and n are independently an integer in a range from 0 to 19,
wherein each of m and n is independently 6, 8, or 10.

11. The glycolipid-heavy metal ion complex of claim 1, wherein R¹ is C₆₋₂₀ alkyl.

12. The glycolipid-heavy metal ion complex of claim 1 further comprising at least one C1 to C6 alcohol associated with the glycolipid-heavy metal ion complex.

13. The glycolipid-heavy metal ion complex of claim 1, wherein the glycolipid-heavy metal ion complex is substantially insoluble at 25° C. at a pH in a range from about 3 to about 12.

14. A method for removing a heavy metal ion from a sample, said method comprising:
contacting said sample with an aqueous solution comprising a glycolipid to form a glycolipid-heavy metal ion complex according to claim 1; and
separating said glycolipid-heavy metal ion complex, thereby removing said heavy metal ion from said sample.

15. The method of claim 14, wherein said sample comprises any of soil, groundwater, industrial wastewater, acid mine drainage, mining process waters and solid residuals, produced waters, electroplating solutions, coal combustion process waters, landfill leachates, e-waste, and fly ash; wherein mining comprises coal and hard rock mining.

16. The method of claim 14, wherein said step of separating said glycolipid-heavy metal ion complex comprises any of ion flotation and/or precipitate flotation, precipitation, centrifugation, filtration, gravitational settling, or combinations thereof.

17. A composition comprising:
at least one glycolipid of the formula:
A-B
wherein
A is selected from the group consisting of a monosaccharide, a disaccharide, and a derivative thereof; and
B is a moiety of the formula: 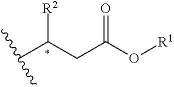
wherein
* is a chiral center;
R² is C₁-C₂₀ hydrocarbon optionally having 1, 2, or 3 carbon-carbon double bonds; and
R¹ is H, C₁-C₂₀ hydrocarbon optionally having 1, 2, or 3 carbon-carbon double bonds, or a moiety of the formula: 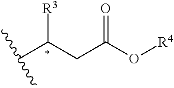
wherein
R³ is C₁-C₂₀ hydrocarbon optionally having 1, 2, or 3 carbon-carbon double bonds, and
R⁴ is H or C₁-C₂₀ hydrocarbon optionally having 1, 2, or 3 carbon-carbon double bonds;
a frothing agent, wherein the frothing agent comprises a C1-C3 alcohol; and
water.

18. The composition of claim 17, wherein a concentration of the glycolipid in the water is in a range of from about 50 μM to about 10 mM.

19. The composition of claim 17 wherein a concentration of the frothing agent in the water is in a range of from about 0% to about 2% (v/v).

20. The composition of claim 17, wherein a ratio of frothing agent to glycolipid is in a range from about 0:1 to about 1500:1.
